(19) United States
(12) Patent Application Publication
(10) Pub. No.: US 2026/0216351 A1
(43) Pub. Date: Jul. 30, 2026

(54) CORRECTION OF ALZHEIMER'S DISEASE PATHOLOGY

(71) Applicant: NeuroTher, LLC, Boston, MA (US)

(72) Inventors: BHANU PRATAP JENA, Bloomfield Hills, MI (US); WON JIN CHO, Newton, MA (US); GUILLERMO G. MARMOL, Boston, MA (US)

(21) Appl. No.: 19/320,987

(22) Filed: Sep. 5, 2025

Related U.S. Application Data
(63) Continuation-in-part of application No. 19/086,505, filed on Mar. 21, 2025.
(60) Provisional application No. 63/751,184, filed on Jan. 29, 2025.

(51) Int. Cl. A61K 47/64; A61P 25/28

(52) U.S. Cl. CPC A61K 47/64; A61P 25/28

(57) ABSTRACT

Disclosed are compositions and methods of use for treating neuronal diseases such as Alzheimer's Disease, Parkinson's Disease, Huntington's Disease, multiple sclerosis, and amyotrophic lateral sclerosis (ALS). In certain embodiments, a flavonoid, such as apigenin, is administered alone or in a pharmaceutical preparation via the nasal olfactory route. In some embodiments, the composition further comprises a porosome complex for reconstitution into neural cells. In additional embodiments, a synthetic blood-brain barrier traversing peptide is included, wherein the peptide is engineered to mimic a domain of ATP1A3 and/or Tubulin, thereby enhancing therapeutic efficacy in restoring secretory and metabolic function in neural tissue.

### CROSS-REFERENCE TO RELATED APPLICATIONS

The instant application is a Continuation-in-Part of U.S. patent application Ser. No. 19/086,505 filed on 21 Mar. 2025, the entirety of which is incorporated by reference herein. The instant application claims the additional benefit of U.S. Provisional Application No. 63/753,184 filed on 29 Jan. 2025, the entirety of which is hereby incorporated by reference.

### SUBMISSION OF SEQUENCE LISTING

The instant application contains an electronically submitted Sequence Listing formatted as a WIPO ST.26 standard.xml file. Said ST.26 file, created on 5 Sep. 2026 is named VRN0012USP and is approximately 2,064 bytes in size and is incorporated by reference herein. The content of the file is the computer readable form (CRF) of one sequence:

SEQ. 1 Illustrates a synthetic DA1 based linear peptide.

### BACKGROUND

### 1. Field of the Discovery

Embodiments of the disclosure presented herein relate generally to the field of neural diseases and their treatments. In some embodiments the neural disease is Alzheimer's Disease (“AD”). Embodiments incorporate the restoration of cell secretory structure function. In other embodiments, a synthetic blood-brain barrier traversing peptide mimicking a specific domain of an impacted porosome protein is administered to restore secretory function in targeted cells.

### 2. Background Information

Porosome organelles are cup-shaped supramolecular lipoprotein structures located at the cell plasma membrane. They are the sites at which secretory vehicles inside the cell transiently dock, fuse, and secret their contents outside the cell.

Typical porosome structures range in size from 15 nm in neurons to 100-180 nm in endocrine and exocrine cells. Porosomes are composed of about 30-40 proteins, with the porosome composition depending on cell type. Porosome-mediated secretion across the cell plasma membrane is a fundamental process through which cells communicate with their environment and exchange information. In a multicellular context, porosome secretion enables cell communities to communicate and maintain homeostasis and, thus, sustain life. Porosomes are present in all secretory cells, from the digestive enzyme-secreting pancreatic acinar cells to the hormone-releasing growth hormone and insulin-secreting cells, mast cells, chromaffin cells, hair cells of the inner ear, and in neurons secreting neurotransmitters. Porosomes have been immunoisolated from a number of cells including the insulin-secreting beta cells of the exocrine pancreas, cells of the human airways' epithelia and neurons, biochemically characterized, and functionally reconstituted into artificial lipid membranes. A large body of evidence has accumulated on the role of porosome-associated proteins on cell secretion and secretory defects, including: neurotransmission and neurological disorders; respiratory disorders; and insulin secretion disorders. Thus, defects in cell secretion stemming from porosome, or porosome component, malfunction are implicated to underpin numerous disease mechanisms including those for cystic fibrosis, diabetes, Alzheimer's, Down Syndrome, schizophrenia, digestive, and immune disorders, among others.

Whereas the “parts list” of biology has been relatively well defined, with many proteins identified in the human cell, a systems level understanding of disease modalities has only begun to be appreciated fully. It is no longer correct to state that any given disease results from a bad copy of a single protein; or, in the face of epigenetics, that expression of a given protein structure results from a single gene alone. In addition, numerous proteins are found to collectively participate in multiple cellular processes (e.g., usually expressed as found in different metabolic or enzymatic pathways), making targeting of single proteins difficult to do without altering multiple cellular functions.

Therefore, targeting a single protein, either for binding or degradation, may treat a disease, but may also inhibit essential bodily functions and/or alternative biochemical pathways that require that same protein. This leads to adverse side effects. Furthermore, each protein requires a “groove” or “binding pocket” for a putative drug to grab onto to bind. Furthermore, this binding pocket on a target protein is also influenced by its neighboring proteins, hence the entire complex needs to be carefully understood for appropriate therapy. Additionally, for small molecules, it is estimated that only 20%-25% of proteins have the necessary groove to bind and be modulated, inhibited or activated.

Alzheimer's disease is a progressive neurodegenerative disorder characterized by cognitive decline and memory loss, largely driven by factors such as amyloid-beta accumulation, tau pathology, neuroinflammation, and oxidative stress. AD is the most common cause of dementia and a leading cause of morbidity and mortality in the aging population. An estimated 6.9 million Americans aged 65 and older are living with AD in 2024, a number that is projected to increase to 13.8 million by 2060. While treatments are available to mitigate AD symptoms, there is no cure currently available. AD inevitably progresses and patients die within an average of 4 to 8 years after AD diagnosis. Therefore, there is an urgent need for novel therapeutic strategies.

In early 2023, there were 187 Phase 1, 2, and 3 clinical trials assessing 141 drugs for AD. Thirty-six drugs were being assessed in Phase 3, 87 in Phase 2, and 31 in Phase 1. Although, neurotransmitter receptors, amyloid plaque, synaptic function, and inflammation were the most common targets of drugs in the pipeline, most therapies have focused on clearing the buildup of amyloid plaques and on reducing inflammation. While these are important advancements in AD therapy, they are primarily focused as treatments to ameliorate the various consequences of the disease; there are no cures in sight, nor are therapies developed that treat the disease at the very early stage.

There is growing evidence that AD is a consequence of metabolic disorder resulting in defects in neurotransmitter release that prevent neurotransmission, leading to a loss of neuronal function and cell death in various regions of the brain. Thus, there is a continued need for treatments that address the observed loss of function and restore neurotransmitter release and subsequent neurotransmission.

### SUMMARY

Disclosed are compositions and/or methods of use of the compositions for patients with neuronal diseases such as AD, Parkinson's, Huntington's, multiple sclerosis, and ALS. In certain embodiments flavonoids alone, or in a pharmaceutical preparation, are administered through the nasal olfactory route. In certain embodiments the flavonoid is apigenin and the neural disease is Alzheimer's. Targeted dosages may affect mitochondrial function in brain neural cells thus ameliorating a neural disease. In some embodiments a porosome complex is administered for reconstitution into a neural cell. In further embodiments, the peptide DA1 may be co-administered with the apigenin.

Embodiments of the disclosure may take the form of a composition. The composition may comprise a ATP1A3-peptide, Tau-binding domain peptide of tubulin, flavonoid and or the porosome in part or whole. The composition may be pharmaceutically acceptable for administration to a subject. In certain embodiments, the ATP1A3-peptide has a sequence mimicking different parts of the extracellular domain of the porosome protein ATP1A3 using reverse engineering to be able to optimally bind and nutralize amyloid beta peptide 1-42. Similarly, the Tau-binding domain peptide of tubulin using reverse engineering to be able to optimally bind and nutralize Tau will be used. In additional embodiments, the composition is a nanoemulsion for nasal administration. In still other embodiments, an additional peptide is a part of the composition. In certain embodiments of the composition the flavonoid is apigenin. In still other embodiments, the composition further contains a porosome complex in par tor whole as applicable.

In one embodiment, a pharmaceutical composition is provided that includes a flavonoid, a synthetic peptide capable of traversing the blood-brain barrier, and a porosome or porosome protein. The composition is formulated in a pharmaceutically acceptable form for treating neuronal disorders. In some embodiments, the blood-brain barrier traversing peptide is configured to mimic a domain of ATP1A3 and/or Tubulin. In some embodiments, the flavonoid is apigenin, and the porosome is a neuronal porosome. In certain embodiments, the synthetic peptide is in a linear or cyclic (macrocyclic) form, such as a DA1 peptide, and the composition may be configured as a nanoemulsion. In some embodiments, the blood-brain barrier traversing peptide and/or the flavonoid is directly linked to the porosome or porosome protein, and additional embodiments further include one or more porosome-associated proteins.

In another embodiment, a method is provided that comprises administering the foregoing composition to a mammal. This treatment is aimed at restoring or correcting cellular secretory and metabolic functions in neural tissue to help ameliorate neurodegenerative diseases. These and other features will be more fully appreciated from the following detailed description and accompanying drawings.

Additional embodiments of the disclosure pertain to a method comprising administering to a subject a composition in accord with any of the embodiments disclosed herein.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated into and form a part of the specification, illustrate several embodiments of the present disclosure; and, together with the description, serve to explain the principles of the disclosure. The drawings are only for the purpose of illustrating an embodiment of the invention and are not to be construed as limiting the disclosure. Further objects, features and advantages of the disclosure will become apparent from the following detailed description taken in conjunction with the accompanying figures showing illustrative embodiments of the invention, in which:

FIG. 1 illustrates an envisioned combination therapy of a neuronal porosome and/or the small flavonoid molecule apigenin.

FIG. 2 Immunoblot and immunocytochemistry demonstrate depletion of porosome proteins SNAP-25, Syntaxin-1A and Na+/K+-ATPase AT1A3, when neurons are exposed to secreted beta amyloid peptide 1-42 in WT-APP and Swe-APP.

FIG. 3 presents example cross-sections of healthy and AD affected human brain sections.

FIG. 4 with sub-figures FIGS. 4a-c present stained postmortem hippocampus sections from control subjects and AD patients. FIGS. 4d-f show quantification of the intensity of SNAP25, Syntaxin 1A, and AT1A3 in NeuN⁺ cells.

FIG. 5 with sub-figures FIGS. 5a-d presents that porosome proteins SNAP-25, Syntaxin-1A and ATP1A3, and the ATAD3A protein essential for mitochondrial fission and bioenergetics, provide superior classification performance in diagnosing AD over other gene products.

FIG. 6 with sub-figures FIGS. 6a-g illustrates the use of AI to reveal altered binding interactions between porosome proteins SNAP-25, Syntaxin-1 and ATP1A3 when amyloid beta 1-42 peptide binding to the extracellular domain of ATP1A3. This binding of the amyloid beta peptide negatively impacts the binding interactions between ATP1A3-SNAP-25-Syntaxin-1A in the cytosolic domain within the neuronal porosome complex.

FIG. 7 with sub-figures FIGS. 7A and 7B, presents high-resolution images of the detailed molecular interaction between ATP1A3 and SNAP 25, with and without Amyloid-β binding.

FIG. 8 with sub-figures FIGS. 8a-f, presents the immunocytochemistry demonstrating depletion of porosome proteins SNAP-25, Syntaxin-1A, and Na⁺/K⁺ Transporting ATPase alpha 3 (AT1A3) in Alzheimer's neurons WT-APP (AB), which is fully restored to normal levels following porosome-reconstitution therapy, and partially by Apigenin and the DA1 peptide.

FIG. 9 with sub-figures FIGS. 9a-f, presents the combinatorial therapy of porosome reconstitution the secretory corrector, and the DA1 peptide inhibitor of mitochondrial fission, the metabolic corrector, restores cell viability and reduces oxidative stress in mitochondria to normal levels in AD neurons. Similarly, combination therapy of the small flavonoid molecule Apigenin and the mitochondrial fission inhibiting linear DA1 peptide results in increase cell viability and reduction of mitochondrial oxidative stress to near normal levels in AD neurons.

FIG. 10 illustrates porosome reconstitution and apigenin treatment overcomes the negative impact of beta amyloid (1-42) on the growth of human brain organoids in culture. There is over a 50% rescue following 15 days after porosome treatment.

FIG. 11 illustrates that a treatment of Alzheimer's mouse hippocampal HT-22 neuronal cells (AB) with porosome reconstitution decreases Tau release.

### DETAILED DESCRIPTION

The disclosed technology relates to compositions and methods for treating neuronal diseases, such as Alzheimer's Disease (AD), by addressing secretory and metabolic dysfunctions. The technical problem is the lack of effective treatments targeting early-stage AD pathology. The solution involves a composition comprising a flavonoid, such as apigenin, a synthetic blood-brain barrier traversing peptide mimicking ATP1A3 and/or Tubulin, and a porosome or porosome protein. This composition is pharmaceutically acceptable and can be administered through the nasal olfactory route to restore cellular secretory and metabolic functions. The primary use is to ameliorate neurodegenerative diseases by enhancing neurotransmitter release and reducing oxidative stress in neural cells. The described technology provides a novel therapeutic strategy for AD, potentially improving patient outcomes by targeting disease mechanisms at an early stage.

Disclosed are compositions and/or methods of use of the compositions for patients with neuronal diseases such as AD, Parkinson's, Huntington's, multiple sclerosis, and amyotrophic lateral sclerosis (ALS). In certain embodiments flavonoids alone, or in a pharmaceutical preparation, are administered through the nasal olfactory route. In certain embodiments the flavonoid is apigenin and the neural disease is Alzheimer's. Targeted dosages may affect mitochondrial function in brain neural cells thus ameliorating a neural disease. In additional embodiments, a porosome complex is administered.

Alzheimer's disease (AD) is characterized by the loss of neurons, as a consequence of secretory and metabolic defects reflected in mitochondrial dysfunction and loss in neurotransmitter release. As herein presented, via application of AlphaFold7 analysis, it is demonstrated how the beta amyloid peptide alters several proteins within the neuronal porosome secretory machinery. This suggests that introduction of functional porosomes into AD neurons would be an effective therapy. The introduction of functional porosomes combined with peptide inhibitors of ATAD3A oligomers restored AD neurons to near normal levels of viability and reduced oxidative stress in mitochondria. The peptide inhibitors of ATAD3A oligomers combined with the flavonoid apigenin restored the viability of AD neurons and reduced mitochondria oxidative stress. Collectively, these results provide an understanding of the molecular underpinnings of porosome dysfunction in AD, enabling the engineering of a new class of AD drugs, and the use of secretory and metabolic correctors for AD therapy.

It has become increasing clear that age and/or diseases contributing to a decline in metabolism (e.g., diabetes), play an important role in the progression of neurodegenerative diseases such as AD. Small molecules that overcome such metabolic decline, therefore, serve as a therapeutic drug to treat AD.

Studies report that the flavonoid apigenin promotes mitochondrial biogenesis by activating the peroxisome proliferator-activated receptor Gamma Co-activator 1-alpha (PGC-1α), prevents neurodegeneration in rat hippocampus, and improves spatial working memory in rats. Apigenin will be used either orally, or via the nasal rout either alone or in combination with peptides targeted toward restoring normal metabolic function by preventing mitochondrial fission, in treating AD and other neurodegenerative diseases.

The unique anatomical and physiological features make it very effective to deliver drugs to the brain through nasal olfactory route, rather than via the systemic circulation. This way, the nasal cavity enables drugs to directly reach the brain, bypassing the blood-brain barrier. The nasal route of drug administration to the brain additionally overcomes metabolism when administered orally, decreases the amount of drug required, and minimizes the compliances associated with injectables into the systemic circulation, providing high pharmacological activity of drugs at lower dosages and with shorter half-lives.

Compelling evidence from basic research and clinical studies indicates that mitochondrial dysfunction is an early prominent event in AD neuropathology including Aβ plaque and neurofibrillary tangles (NFTs) and plays important roles in the pathogenesis of AD. Patients with AD are known to display diminished glucose utilization, abnormal brain energetics, mitochondrial DNA (mtDNA) lesion, and reduced activity of certain enzymes associated with mitochondrial respiratory chain complexes. Decreased ATP and respiratory activity, elevated mitochondrial reactive oxygen species (ROS), enhanced mitochondrial depolarization, and increased mtDNA depletion were also documented in various AD rodent models before disease onset, suggesting that mitochondrial dysfunction is an early event during AD progression. A direct interaction between AB and mitochondria is a relevant component of AD pathology. AB progressively accumulates within mitochondria in the brains of AD patients and rodent AD animals. The key components of the amyloid precursor protein (APP) processing and the β-amyloid producing γ-secretase complex, are highly enriched in the mitochondria, which results in local production of Aβ in the mitochondria. As a consequence, accumulation of Aβ in mitochondria disrupts mitochondrial bio-energetic activity, induces mitochondrial genome instability, increases ROS, and reduces protein degradation capacity, leading to programmed cell death, synaptic defects, and disease-associated pathology. In addition, localization of Tau within mitochondria or its association with the mitochondrial outer membrane has been documented. Tau positive neurons show marked mitochondrial loss. Pathological Tau has been reported to impair mitochondrial trafficking, mitochondrial dynamics and mitophagy. Thus, mitochondria might be an important route through which tau leads to the degeneration and death of neuronal cells. Eliminating damaged mitochondria altogether might be of importance to reduce AD pathology and related cognitive deficiency.

ATAD3A (ATPase family AAA-domain containing protein 3A) is a nuclear-encoded mitochondrial protein belonging to a family of AAA-ATPase proteins specific to multicellular eukaryotes. ATAD3A is a component of the mitochondrial nucleoid complex, which is required for mtDNA nucleoid maintenance. ATAD3A is structurally unique; its C-terminus, which contains the conserved ATPase, is located in the mitochondrial matrix, whereas its N-terminus has been found to be exposed to the cytosol and associate with the mitochondrial outer membrane. Remarkably, ATAD3A contains multiple transmembrane (TM) domains that allow it to traverse both inner-(IMM) and outer-mitochondrial membranes (OMM), at the IMM-OMM contact sites. ATAD3A has been found to regulate mitochondrial morphology and control cholesterol channeling for steroidogenesis at mitochondrial contact sites. Thus, ATAD3A, by straddling the two mitochondrial membranes, simultaneously regulates mitochondrial membrane integrity and mtDNA nucleoid organization.

The expression of mutant ATAD3A in Drosophila causes severe mitochondrial fragmentation, aberrant cristae, and increased mitophagy in both neurons and muscle, leading to early lethality. Although, global ATAD3A knockout (KO) is embryonic lethal, selective ATAD3A KO in mouse causes mitochondrial fragmentation and mitochondrial bioenergetics failure, resulting in cell death and tissue damage. ATAD3A mutation is also associated with axonal neuropathy and spastic paraplegia. Therefore, proper function of ATAD3A is crucial for mitochondrial activity, integrity and cellular survival. In AD, ATAD3A undergoes oligomerization, resulting in Drp1-mediated mitochondrial fragmentation, leading to neurodegeneration. Heterozygous knockdown of ATAD3A in AD transgenic mice abolishes ATAD3A oligomerization, reduces amyloid accumulation and neuroinflammation, and improves long-term memory.

Patients carrying a recently identified ATAD3A mutant show neurodegenerative conditions associated with developmental delay, axonal neuropathy, and spastic paraplegia. These data further support the idea that aberrant ATAD3A oligomerization is a key pathogenic factor to induce AD neuropathology and further validates ATAD3A as a drug target. Thus, embodiments of the disclosure deliver ATAD3A affecting peptides as part of a treatment for one or more neurological disorders.

In embodiments as used herein DA1 refers to a peptide corresponding to a homologous region between Drp1 and ATAD3A as known in the art. The sequence of DA1 is known to be highly conserved between species. SEQ. ID. NO: 1 presents an example sequence of a DA1 peptide: EDKRKT. Those of skill in the art can appreciate that conservative amino acid substitutions may be made as necessary. Those of skill in the art can further recognize that in certain circumstances one or more additional peptides of different sequences may also correspond to a homologous region between Drp1 and ATAD3A, such variants are known and contemplated as within the scope of this disclosure.

Porosome organelles, or complexes, are cup-shaped supramolecular lipoprotein structures located at the cell plasma membrane. They are the sites at which secretory vesicles inside the cell transiently dock, fuse, and secret their contents outside the cell. They are the universal secretory machinery of the cell. The porosome structure includes multiple proteins with their associated ligands, chaperones, and other affiliated molecules such as lipids. Although it is classically understood that there are diseases caused by mutations/malformations in the structure of a single protein, it is only recently starting to be understood that malfunction and malformations of larger structures, such as the porosome, contribute to diseases.

Porosomes enable communication (language) between cells in the body by secreting chemical messages such as neurotransmitters from nerve cells or hormones from endocrine cells such as insulin from B-cells of the endocrine pancreas. These chemical messages are stored in secretory vesicles within the cell. The porosome secretory machinery, composed of 30+ proteins, provides instructions to secretory vesicles to appropriately dock at the porosome base, fuse, swell and release measured amounts of intra-vesicular contents to the outside. None of the individual components of the porosome are unique to it. Rather, the combination of 30 or more proteins together in the proper conformation within the complex, provides function to the structure. Thus, envisioned embodiments of this disclosure entail correcting cellular function by targeting a specific multimer, such as the porosome complex, without altering the activity of other cellular complexes, hence other cellular processes that possess one or more of the individual components of the targeted structure.

Typical porosome structures range in size from 15 nm in neurons to 100-180 nm in endocrine and exocrine cells. Porosomes are composed of about 30-40 proteins, with the porosome composition depending on cell type. Porosome-mediated secretion across the cell plasma membrane is a fundamental process through which cells communicate with their environment and exchange information. In a multicellular context, porosome secretion enables cell communities to communicate and maintain homeostasis and, thus, sustain life. Porosomes are present in all secretory cells, from the digestive enzyme-secreting pancreatic acinar cells to the hormone-releasing growth hormone and insulin-secreting cells, mast cells, chromaffin cells, hair cells of the inner ear, and in neurons secreting neurotransmitters. Porosomes have been immunoisolated from a number of cells including the insulin-secreting beta cells of the exocrine pancreas, cells of the human airways epithelia and neurons, biochemically characterized, and functionally reconstituted into artificial lipid membranes. A large body of evidence has accumulated on the role of porosome-associated proteins on cell secretion and secretory defects, including in neurotransmission and neurological disorders. Thus, defects in cell secretion stemming from porosome, or porosome component, malfunction are implicated to underpin numerous disease mechanisms, including those in many neurological disorders.

Tables containing lists of porosome proteins and protein-protein interactions are known and widely available to those of skill in the art. As used herein, a “porosome protein” is one that is a part, either singly or in multiple copies, of the overall porosome structure. Those of skill in the art can readily appreciate that porosomes associated with different cell/tissue types may have various proteins that compose them. As used herein, a “porosome-associated protein” is one that is found to interact with a porosome protein, or to interact with a porosome structure.

In an embodiment of the disclosure an entire functional porosome can be reconstituted into a targeted tissue such as, for instance, in the neural cells of a patient experiencing a neurological disease. In some embodiments, for the reconstitution, a porosome from pig or human sources is extracted and put in a human cell. In still other embodiments, the nanoscale porosome complex for reconstitution is obtained from CALU 3 or human neural cells to address the neural disease. Reconstitution therapy involves reconstituting or introducing a normal functional porosome complex at the cell plasma membrane of the neurons in patients experiencing a neurological disorder. Without subscribing to, or being bound by, a particular theory, reconstitution of a prosome complex coupled with a peptide inhibitor of mitochondrial fission, provided and/or dosed in sufficient amounts are sufficient to treat, ameliorate symptoms, or otherwise alter the course of a neurological disorder. It is known to practitioners that porosomes reconstituted into live cells are stable and functional. In certain embodiments the porosome and/or a porosome containing complex (e.g., a porosome bound to one or more peptides) or mixture (e.g., containing a flavonoid or peptide) may be additionally packaged for introduction to a cellular membrane. Packaging in this sense may mean a coating, such as with lipids or within a lipid membrane, and/or the usage of one or more pharmaceutical excipients (e.g., such as in a mist or nanoemulsion).

In AD, the proteins 2,3-cyclic nucleotide phosphodiesterase (CNPase) and the heat shock protein 70 (HSP70) are implicated as playing a role in disease pathology. The levels of CNPase and HSP70, both present in the neuronal porosome complex are found to increase, while the levels of porosome-associated dihydropyrimidinase-related protein-2 (DRP-2) is decreased. Similarly, porosome proteins SNAP-25 and synaptophysin are significantly reduced in neurons of patients with AD.

Decreased levels of CNPase have also been reported in the frontal and temporal cortex of patients with AD and Down syndrome. Low CNPase levels have also been detected in the anterior frontal cortex in schizophrenic patients. Additionally, an allele that is associated with low levels of CNPase is also reported to be linked to Schizophrenia.

Examples of neuronal porosome proteins can include: Tubulin beta, myosin 7b, spectrin, Creatine kinase, Dystrophin, Langerin, GTPase activating protein (GAP), Intersectin 1 isoform (ITSN-1), Actin, cytoplasmic 1, Sodium/potassium-transporting ATPase subunit alpha-3, Plasma membrane calcium-transporting ATPase 1, Plasma membrane calcium-transporting ATPase 2, Brain acid soluble protein 1, Adenylyl cyclase-associated protein 1, 2′,3′-Cyclic-nucleotide 3′-phosphodiesterase, Dihydropyrimidinase-related protein 2, Dihydropyrimidinase-related protein 3, Dihydropyrimidinase-related protein 5, Glutamine synthetase, Guanine nucleotide-binding protein G (o) subunit alpha, Neural cell adhesion molecule 1, Vesicle-fusing ATPase, Ras-related protein Rab-3A, Reticulon-3, Reticulon-4, Synaptosomal-associated protein 25, Syntaxin-1A, Syntaxin-1B, Syntaxin-binding protein 1, Synapsin-2, Synaptophysin, Synaptotagmin-1, Tubulin alpha-1A chain, Vesicle-associated membrane protein 1, Vesicle-associated membrane protein 2, V-type proton ATPase subunit B, brain isoform. Embodiments of the invention can include one or more identified small molecules that directly act upon one or more of the above proteins to affect neuronal porosome structure and/or function.

Small molecule inhibitors & stimulators of porosome phosphodiesterase such as Vinpocetine, BAY 60-7550, Rolipram, Etazolate, Sildenafil, S14, VP1.15, PF-04447943, Papaverine, and the small molecule inhibitors Apoptozole, VER155008, JG98, HA15 and YUM70 of HSP70, and small molecule activator ML346 for HSP70, all may be used to treat neuronal diseases especially Alzheimer's.

In some embodiments, the porosome complex is isolated from cells or cell lysate obtained from a mammalian cell. In some instances, the mammalian cell is an epithelial cell, connective tissue cell, hormone secreting cell, a nerve cell, a skeletal muscle cell, a blood cell, or an immune system cell. In certain embodiments the cell may one sampled from a subject and subsequently proliferated.

Exemplary mammalian cells include, but are not limited to, 293A cell line, 293 FT cell line, 293F cells, 293 H cells, HEK 293 cells, CHO DG44 cells, CHO-S cells, CHO-K1 cells, Expi293F™ cells, Flp-In™ T-REX™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REX™ Jurkat cell line, Per.C6 cells, T-REX™-293 cell line, T-REX™-CHO cell line, T-REX™-HeLa cell line, NC-HIMT cell line, and PC12 cell line.

In some instances, the porosome containing cell sample or cell lysate sample is obtained from cells of a tumor cell line. In some instances, the cell sample or cell lysate sample is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the porosome containing cell sample or cell lysate sample is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some instances, the hematologic malignant cell line is obtained from a B-cell cell line of: acute lymphoblastic leukemia (ALL), acute myelogenous leukemia (AML), chronic myelogenous leukemia (CML), acute monocytic leukemia (AMOL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the porosome containing cell sample or cell lysate sample is obtained from a tumor cell line. Exemplary tumor cell line includes, but is not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, the porosome containing cell sample or cell lysate sample is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, the cell sample or cell lysate sample is a tissue sample, such as a sample obtained from a biopsy or a tumor tissue sample. In some embodiments, the cell sample or cell lysate sample is a blood serum sample. In some embodiments, the cell sample or cell lysate sample is a blood cell sample containing one or more peripheral blood mononuclear cells (PBMCs). In some embodiments, the cell sample or cell lysate sample contains one or more circulating tumor cells (CTCs). In some embodiments, the cell sample or cell lysate sample contains one or more disseminated tumor cells (DTC, e.g., in a bone marrow aspirate sample).

In some embodiments, the porosome containing cell sample or cell lysate sample is obtained from an individual by any suitable means of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, would be examined under a microscope.

Neurotransmitter Release: As above mentioned, porosomes are secretory portals at the cell plasma membrane where secretory vesicles transiently dock and fuse to expel a precise amount of intra-vesicular contents from the cell during secretion. In neurons, porosomes are 15 nm cup-shaped lipoprotein structures at the presynaptic membrane, composed of nearly 40 proteins. A number of porosome proteins have previously been implicated in neurotransmission and neurological disorders, attesting to the crosstalk between porosome proteins and their coordinated involvement in release of neurotransmitter at the synapse. In AD, levels of porosome proteins CNPase (2,3-cyclic nucleotide phosphodiesterase) and the heat shock protein 70 (HSP70) are found to increase, while the levels of dihydropyrimidinase-related protein-2 (DRP-2) decrease. Decreased levels of CNPase have been observed in the frontal and temporal cortex of patients with AD. Similarly, porosome proteins SNAP-25 and synaptophysin are significantly reduced in neurons of patients with AD. Mice that are SNAP-25 (+/−) show disabled learning and memory, and exhibit epileptic like seizures. These results support that, alteration of one porosome protein impacts others within the complex, resulting in impaired porosome-mediated secretion. This is similar to recent studies on human bronchial epithelial (HBE) cells showing that HBE cells with AF508 cystic fibrosis transmembrane conductance regulator (CFTR) mutation, affects nearly a dozen porosome proteins including CFTR within the porosome complex. Therefore, the reprogramming of the porosome secretory machinery into the cell plasma membrane of Cystic Fibrosis (CF) cells, was able to rescue from CF. Thus, embodiments of the disclosure herein support that reconstitution of the 15 nm normal neuronal porosome complex in AD neurons overcome secretory defects in neurotransmitter release.

Neuronal Energy Metabolism: It is widely accepted that mitochondrial production of reactive oxygen species (ROS) contributes to the detrimental alterations in the etiology and/or progression of many pathological conditions, including brain neurodegeneration. Studies report that flavonoids can protect cells from different insults that lead to mitochondria-mediated cell death, and epidemiological data further show that some of these compounds attenuate the progression of diseases associated with oxidative stress and mitochondrial dysfunction. Flavonoids are low molecular weight phenolic compounds, displaying significant ROS scavenging capability, including other cellular antioxidant effects, hence ideal for the protection of brain neurons from buildup of free radicals generated by the mitochondria leading to mitochondrial fission in AD.

While various embodiments of the present disclosure are described herein, it will be understood by those skilled in the art that such embodiments are provided by way of example only. It will be understood by those skilled in the art that numerous modifications and changes to, and variations and equivalent substitutions of, the embodiments described herein can be made without departing from the scope of the disclosure. It is understood that various alternatives to the embodiments described herein may be employed in practicing the disclosure, and modifications may be made to adapt a particular structure or material to the teachings of the disclosure. It is also understood that every embodiment of the disclosure may optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

Unless otherwise defined, all technical and scientific terms used herein have the same meaning as commonly understood by one of ordinary skill in the art to which this disclosure belongs.

Where a range of values is provided, it is understood that each intervening value between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding both of those included limits are also included in the disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The term “exemplary” as used herein means “serving as an example, instance or illustration”. Any embodiment or feature characterized herein as “exemplary” is not necessarily to be construed as preferred or advantageous over other embodiments or features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

Apigenin (4′,5,7-trihydroxyflavone; CAS NO: 520-36-5), found in many plants, is a natural product belonging to the flavone class that is the aglycone of several naturally occurring glycosides. It is a yellow crystalline solid that has been used to dye wool.

Pharmaceutical preparations or compositions described or used herein may further comprise coloring or stabilizing agents, osmotic agents, antibacterial agents, or any other substances as long as such substances do not interfere with the function of the composition. The pharmaceutical compositions of the instant disclosure, can, for example, be formulated as a solution, suspension, or emulsion in association with a pharmaceutically acceptable parenteral vehicle. Examples of such vehicles are water, saline, Ringer's solution, dextrose solution, and 5% human albumen. Liposomes may also be used. The vehicle may contain additives that maintain isotonicity (e.g., sodium chloride or mannitol) and chemical stability (e.g., buffers and preservatives). It should be appreciated that endotoxin contamination should be kept at a safe level, for example, less than 0.5 ng mg⁻¹ protein. Moreover, for human administration, preparations should meet sterility, pyrogenicity, general safety and purity standards as required by the United States Food and Drug Administration Office of Biological Standards. The formulations may be sterilized by commonly used techniques such as filtration.

The phrase “pharmaceutically acceptable” refers to substances and compositions which do not produce an adverse, allergic, or otherwise untoward reaction when administered to an animal, or a human, as appropriate. A substance which caused or produced any of these adverse effects would be classified as “biologically harmful’ within the scope of the present disclosure. Pharmaceutically acceptable substances and compositions include, but are not limited to solvents, dispersion media, coatings, antibacterial and antifungal agents, isotonic and absorption delaying agents. Except where incompatible with the disclosure the use of any conventional ingredient is contemplated. Furthermore, supplementary active ingredients which serve some other pharmacologically expedient purpose can also be incorporated into the instant compositions without departing from the broader scope of the instant disclosure.

The effective dose and method of administration of a particular embodiment of the instant disclosure may vary based on the individual patient and stage of any present diseases (e.g., breast cancer, HIV, other co-morbidities), as well as other factors known to those of skill in the art. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

Toxicity and safe dosage levels may be determined through the determination of dose-limiting toxicities (DLTs) and the overall DLT-rate (e.g., such as in the context of a clinical trial). In certain embodiments compositions with a DLT rate less than 25% are considered safe.

Effectiveness of embodiments disclosed herein may further be evaluated through cohort studies and examination of the recurrence-free survival (RFS) rate at chosen time intervals.

An “effective amount” of an agent or therapeutic peptide is an amount sufficient to achieve a desired therapeutic or pharmacological effect, such as an amount that is capable of activating the growth of neurons. An effective amount of an agent as defined herein may vary according to factors such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

The exact dosage is chosen by an individual physician in view of a patient to be treated. Dosage and administration are adjusted to provide sufficient levels of embodiments of the instant disclosure to maintain the desired effect (e.g., inducement of an immune response against uveal melanoma). Additional factors that may be taken into account include the severity of any disease state, age, weight, and gender of the patient; diet, time and frequency of the administration, drug combination(s), reaction sensitivities, and tolerance/response to therapy. Short acting pharmaceutical compositions are administered daily whereas long-acting pharmaceutical compositions are administered every 2, 3 to 4 days, every week, or once every two weeks or more. Depending on half-life and clearance rate of the particular formulation, the pharmaceutical compositions of the instant disclosure may be administered once, twice, three, four, five, six, seven, eight, nine, ten or more times per day.

Normal dosage amounts may vary from approximately 1 to 100,000 micrograms, up to a total dose of about 10 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5 g, 6 g, 7 g, 8 g, 9 g, and 10 g.

More specifically, the dosage of peptide agents described herein is one that provides sufficient peptide agent to attain a desirable effect, including stimulation of the immune system to induce a treatment effect or effecting the functioning of cellular mitochondria. Accordingly, the dose of the peptide agent preferably produces a tissue or blood concentration of both about 1 to 800 μM. Preferable doses produces a tissue or blood concentration of greater than about 10 μM to about 500 μM. Preferable doses are, for example, the amount of peptide required to achieve a tissue or blood concentration or both of 10 μM, 15 μM, 20 μM, 25 μM, 30 μM, 35 μM, 40 μM, 45 μM, 50 μM, 55 μM, 60 μM, 65 μM, 7 μM, 75 μM, 80 μM, 8 μM, 90 μM, 95 μM, 100 μM, 110μ, 120μ, 130μ, 140μ, 150μ, 160μ, 170μ, 180μ, 190μ, 200μ, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. Although doses that produce a tissue concentration greater than 800 μM are not necessarily preferred, they are envisioned and can be used with some embodiments of the present disclosure. A constant or cyclic infusion of embodiments of the disclosure can be provided to maintain a stable concentration of the therapeutic agents.

The pharmacologically active compounds of this invention can be processed in accordance with conventional pharmaceutical practices to produce medicinal agents for administration to patients (e.g., mammals, including humans). The peptides with, or without, modification can be incorporated into a pharmaceutical composition. Further, the manufacture of pharmaceuticals or therapeutic agents that deliver the peptides or a nucleic acid sequence encoding a peptide by several routes is an embodiment.

The term “administering” or “administer” to a patient includes dispensing, delivering or applying an active compound in a pharmaceutical formulation to a subject by any suitable route for delivery of the active compound to the desired location in the subject (e.g., to thereby contact a desired cell, such as a desired neuron), including administration into the cerebrospinal fluid or across the blood-brain barrier, delivery by either the parenteral or oral route, intramuscular injection, subcutaneous or intradermal injection, intravenous injection, buccal administration, transdermal delivery and administration by the rectal, colonic, vaginal, intranasal or respiratory tract route. The agents may, for example, be administered to a comatose, anesthetized, or paralyzed subject via an intravenous injection or may be administered intravenously to a pregnant subject to stimulate axonal growth in a fetus. Specific routes of administration may include topical application (such as by eyedrops, creams or erodible formulations to be placed under the eyelid, intraocular injection into the aqueous or the vitreous humor, injection into the external layers of the eye, such as via subconjunctival injection or subtenon injection, parenteral administration or via oral routes.

As used herein the term “sequence” explicitly contemplates DNA, cDNA, RNA and resulting peptide chains encoded thereby in both sense and antisense directions. To know one is to know the others via the standard rules of complementarity and codon encoding as exemplified in standardized DNA, RNA, and amino acid codon tables.

A “peptide” in the context of the present disclosure is to be understood as meaning a polymer composed of amino acids, preferably the 20 proteinogenic L-amino acids, preferably of linear structure, which has up to 100 amino acids which are linked to one another via peptide bonds. According to the disclosure, the peptides of the disclosure have an amino acid sequence of 4 to 50 amino acids. In the context of this disclosure, the amino acids are given in a one-letter code, where, for example, C stands for cysteine, R for arginine, A for alanine and L for leucine. It is further understood that unless otherwise indicated, the amino acids in an amino acid sequence disclosed herein are linked via peptide bonds and, unless otherwise indicated, the sequence is listed in N- to C-terminal orientation.

Peptides can be chemically synthesized in various embodiments and/or recombinantly produced using protein design. Short peptides can easily be prepared synthetically, for example via solid phase synthesis. Longer peptides and polypeptides, on the other hand, are often produced recombinantly in A host organism.

Typical acidic or negatively charged amino acids (depending on pH) are D and E.

The positively charged or basic amino acids (depending on the pH value) typically include R, K and H.

Amino acids such as G, A, C, I, L, M, F, V, P, S, T, W, Y, N and Q are typically uncharged, i.e., neutral, amino acids.

When reference is made herein to an “any” amino acid, what is commonly meant is one of the 20 naturally occurring proteinogenic amino acids, i.e. one of glycine (G), alanine (A), valine (V), leucine (L), isoleucine (I), Phenylalanine (F), Serine(S), Threonine (T), Proline (P), Methionine (M), Cysteine (C), Histidine (H), Lysine (K), Arginine (R), Glutamine (Q), asparagine (N), aspartic acid (D), glutamic acid (E), tyrosine (Y) and tryptophan (W). Unless otherwise stated, the amino acids are typically L-amino acids. In alternative embodiments, the peptide can also consist of D-amino acids, although it may be preferred that D- and L-amino acids do not occur at the same time within the peptides described herein. In various embodiments, any such amino acid includes all the aforementioned amino acids with the exception of proline, or in some embodiments also with the exception of proline and glycine.

The identity of nucleic acid or amino acid sequences is determined by sequence comparison. This sequence comparison is based on the BLAST algorithm established and commonly used in the prior art (cf. e.g., Altschul et al. (1990) Basic local alignment search tool, J. Mol. Biol., 215:403-410, and Altschul et al. (1997) Gapped BLAST and PSI-BLAST: a new generation of protein database search programs, Nucleic Acids Res., 25:3389-3402) and basically happens by similar sequences of nucleotides or amino acids in the nucleic acid or amino acid sequences be assigned. A tabular assignment of the relevant positions is called alignment. Another algorithm available in the art is the FASTA algorithm. Sequence comparisons (alignments), especially multiple sequence comparisons, are created using computer programs. For example, the Clustal series (see e.g., Chenna et al. (2003) Multiple sequence alignment with the Clustal series of programs, Nucleic Acid Res., 31:3497-3500), T-Coffee (see e.g., Notredame et al. (2000) T-Coffee: A novel method for multiple sequence alignments, J. Mol. Biol., 302:205-217) or programs based on these programs or algorithms. Sequence comparisons (alignments) are also possible using the computer program Vector NTI® Suite 10.3 (Invitrogen Corporation, 1600 Faraday Avenue, Carlsbad, California, USA) with the specified standard parameters, whose AlignX module for sequence comparisons is based on ClustalW, or Clone Manager 10 (Use of the BLOSUM 62 scoring matrix for sequence alignment at the amino acid level). Unless otherwise stated, sequence identity reported herein is determined using the BLAST algorithm.

Such a comparison also allows a statement to be made about the similarity of the compared sequences to one another. It is usually given as percent identity, i.e., the proportion of identical nucleotides or amino acid residues in the same positions or in positions corresponding to one another in an alignment. The broader concept of homology includes conserved amino acid exchanges in amino acid sequences, i.e., amino acids with similar chemical activity, since these usually exert similar chemical activities within the protein. Therefore, the similarity of the compared sequences can also be stated as percent homology or percent similarity. Identity and/or homology information can be made for entire polypeptides or genes or just for individual regions. Homologous or identical regions of different nucleic acid or amino acid sequences are therefore defined by matches in the sequences. Such areas often have identical functions. They can be small and contain only a few nucleotides or amino acids. Such small areas often perform essential functions for the overall activity of the protein. It can therefore make sense to relate sequence matches only to individual, possibly small areas. Unless otherwise stated, identity or homology information in the present application refers to the total length of the nucleic acid or amino acid sequence specified in each case.

The peptide or protein concentration can be determined using known methods, for example the BCA method (bicinchoninic acid; 2,2′-biquinolyl-4,4′-dicarboxylic acid) or the biuret method (Gornall et al., J. Biol. Chem., 1948, 177:751-766). Those skilled in the art of peptide and protein technology will be aware of a variety of suitable methods for determining peptide or protein concentration that can be used within the scope of this disclosure.

Peptides according to the disclosure can have amino acid changes, in particular amino acid substitutions, insertions, or deletions. Such peptides are further developed, for example, through targeted genetic modification, i.e., through mutagenesis processes, and optimized for specific purposes or with regard to special properties (e.g., in terms of their stability, binding, etc.).

For example, targeted mutations such as substitutions, insertions or deletions can be introduced into the known molecules in order to change certain properties, for example. For this purpose, in particular the surface charges and/or the isoelectric point of the molecules and thereby their interactions with a surface can be changed. For example, the net charge of the peptides can be changed in order to influence substrate binding. Alternatively, or additionally, one or more corresponding mutations can, for example, increase the stability or adsorption of the peptide. Advantageous properties of individual mutations, e.g., individual substitutions, can complement each other.

The term “conservative amino acid substitution” means the exchange (substitution) of an amino acid residue for another amino acid residue, whereby this exchange does not lead to a change in polarity or charge at the position of the exchanged amino acid, e.g., the exchange of a non-polar amino acid residue for another non-polar amino acid residue. Conservative amino acid substitutions within the scope of the disclosure include, for example: G=A=S, I=V=L=M, D=E, N=Q, K=R, Y=F, S=T, G=A=I=V=L=M=Y=F=W=P=S=T.

In preferred embodiments, the peptide according to the disclosure can also be modified. Preferred modifications can be, for example, coupling the peptide with certain other molecules or chemical groups, for example organic (macro) molecules, for example via a covalent bond or a linker/spacer via a suitable amino acid of the chain and/or N- and/or C-terminal.

All the aforementioned features and embodiments can be implemented individually or in any combination.

Furthermore, the peptide according to the disclosure can also be at least one subunit (module) of a larger peptide or polypeptide, where the polypeptide can comprise a multimer of the sequences described herein, for example 1 to 30 repeats, more preferably 2 to 15 repeats, particularly preferably 2 to 10 repeats, e.g., 2, 3, 4, 5 or 6 repeats of the peptide. The polypeptide may include or consist of such multimers. The term “polypeptide” in this context refers in particular to those peptides that comprise 100 or more amino acids. The term “larger peptides” preferably refers to peptides with at least 40 amino acids, unless otherwise described.

In various embodiments, the peptide is a peptide or polypeptide (multimer) comprising two or more of the peptides as described herein. In various embodiments, the two or more peptides can be connected to one another by at least one spacer, preferably the at least one spacer comprises or consists of 1 to 10 amino acid residues, in particular 2, 3 or 4 amino acid residues, preferably selected from the group consisting of G, P, I, A and S or combinations thereof, in particular GPI or GAS. In such embodiments, the individual peptides are optionally connected linearly to one another via peptide bonds, possibly also via a spacer.

The peptides described herein may have been chemically synthesized in various embodiments and/or recombinantly produced using protein design. Nowadays, short peptides can easily be prepared synthetically, for example using solid-phase synthesis such as Merrifield's solid-phase synthesis. Longer peptides and polypeptides, on the other hand, are often produced recombinantly in the host organism, e.g., in bacteria or yeast.

It is preferred to produce the peptides and/or peptide conjugates according to the disclosure using recombinant processes. This includes all genetic engineering or microbiological processes that are based on the genes for the peptides of interest being introduced into a host organism suitable for production and transcribed and translated by it (summarized in the context of this disclosure as biotechnological processes).

The peptides and/or peptide conjugates according to the disclosure may be produced as polypeptides (multimers) and subsequently cleaved into the functional peptides and/or peptide conjugates. Very particularly preferred multimers have 1 to 30 peptide units (each according to the disclosure), each of which is separated from one another by spacers of 1 to 10 amino acids long (e.g., 1, 2, 3 or 4 amino acids). Alternatively, the spacers can also be or include interfaces for specific proteases/peptidases, in particular endopeptidases, or can form such an interface together with parts of the peptide.

Using methods that are generally known today, such as chemical synthesis or the polymerase chain reaction (PCR) in conjunction with standard molecular biological and/or protein chemical methods, it is possible for a person skilled in the art to identify the corresponding nucleic acids and even complete genes using known DNA and/or amino acid sequences to produce. Such methods are, for example, from Sambrook, J., Fritsch, E. F. and Maniatis, T. 2001. Molecular cloning: a laboratory manual, 3rd Edition Cold Spring Laboratory Press. known.

In embodiments, peptides and/or peptide conjugates described herein are produced using biotechnological processes as described herein and/or are ordinarily known to those of skill in the art.

The term “expression” refers to the process by which nucleic acid is translated into peptides or is transcribed into RNA, which, for example, can be translated into peptides, polypeptides, or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

The terms “derived from” or “based on” in reference to peptide, amino acid, and nucleic acid sequences are viewed as standard terms of source attribution that may then be varied. For example, an RNA sequence may be derived from a DNA sequence and vice versa. Likewise, a peptide or amino acid sequence may be provided and a subsequent sequence based on that reference sequence created. Thus, to know that source or reference sequence is to have the basic element that may then be varied.

The term “heterologous nucleic acid sequence” is typically DNA that encodes RNA and proteins that are not normally produced in vivo by the cell in which it is expressed or that mediates or encodes mediators that alter expression of endogenous DNA by affecting transcription, translation, or other regulatable biochemical processes. A heterologous nucleic acid sequence may also be referred to as foreign DNA. Any DNA that one of skill in the art would recognize or consider as heterologous or foreign to the cell in which it is expressed is herein encompassed by heterologous DNA. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies. Antibodies that are encoded by heterologous DNA may be secreted or expressed on the surface of the cell in which the heterologous DNA has been introduced.

The terms “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

The term “nanoemulsion” (sometimes known as a “miniemulsion” by those of skill in the art) is a heterogeneous formulation of two different immiscible liquids (e.g., oil and water), often stabilized by surface-active agents (e.g., surfactants) to produce droplets within the nano-range (20-200 nm). Pharmaceutical nanoemulsions can be administered by SC, IM, intravenous, nasal, and mucosal routes.

The term “patient” or “subject” or “animal” or “host” refers to any mammal. The subject may be a human; but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

The terms “prevent” or “preventing” refer to reducing the frequency or severity of a disease or condition. The term does not require an absolute preclusion of the disease or condition. Rather, this term includes decreasing the chance for disease occurrence.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The agents, compounds, compositions, antibodies, etc. used in the methods described herein are considered to be purified and/or isolated prior to their use. Purified materials are typically “substantially pure”, meaning that a nucleic acid, polypeptide or fragment thereof, or other molecule has been separated from the components that naturally accompany it. Typically, the polypeptide is substantially pure when it is at least 60%, 70%, 80%, 90%, 95%, or even 99%, by weight, free from the proteins and other organic molecules with which it is associated naturally. For example, a substantially pure polypeptide may be obtained by extraction from a natural source, by expression of a recombinant nucleic acid in a cell that does not normally express that protein, or by chemical synthesis. “Isolated materials” have been removed from their natural location and environment. In the case of an isolated or purified domain or protein fragment, the domain or fragment is substantially free from amino acid sequences that flank the protein in the naturally-occurring sequence. The term “isolated DNA” means DNA has been substantially freed of the genes that flank the given DNA in the naturally occurring genome. Thus, the term “isolated DNA” encompasses, for example, cDNA, cloned genomic DNA, and synthetic DNA.

The terms “portion”, “fragment”, “variant”, “derivative” and “analog”, when referring to a polypeptide include any polypeptide that retains at least some biological activity referred to herein (e.g., inhibition of an interaction such as binding). Polypeptides as described herein may include portion, fragment, variant, or derivative molecules without limitation, so long as the polypeptide still serves its function. Polypeptides or portions thereof of the present invention may include proteolytic fragments, deletion fragments, or fragments that more easily reach the site of action when delivered to an animal.

Embodiments described herein relate to methods of inhibiting ATAD3A oligomerization, ATAD3A activation, and/or Drp1 activation in cells (e.g., nerve cells) of subjects with neurodegenerative disorders associated with aberrant ATAD3A activation and particularly relates to methods of treating disorders and/or neurodegenerative disorders associated with aberrant ATAD3 A activation in a subject.

ATAD3A is a nuclear-encoded mitochondrial protein that spans the inner and outer membranes with its two terminal domains located in the outer membrane and the matrix. ATAD3A regulates mitochondrial morphology and controls cholesterol trafficking at mitochondrial contact sites. Either overexpression or downregulation of ATAD3A results in mitochondrial fragmentation, suggesting a scaffold-like property on maintenance of mitochondrial morphology. Moreover, ATAD3A is a component of mitochondrial nucleoid complex, which implicates in mtDNA nucleoid maintenance. While global knockout of ATAD3A is embryonic lethal, selective loss of ATAD3 A in mouse skeletal muscle disrupts mitochondrial ultrastructure and reduces the number of cristae junctions, which impairs mtDNA integrity. The expression of mutant ATAD3A in Drosophila causes severe mitochondrial fragmentation, aberrant cristae, and increased mitophagy in both motor neurons and muscle, leading to early lethality. Patients carrying an ATAD3A mutant show neurodegenerative conditions associated with axonal neuropathy, and spastic paraplegia. The proper function of ATAD3A is therefore critical for cell survival.

### Selected Abbreviations

Aβ amyloid beta
AD Alzheimer's Disease
ATAD3A ATPase family AAA-domain containing protein 3 A
BBB blood brain barrier
Cmax maximum concentration
CNS central nervous system
DA1 peptide corresponding to the Drp1 region of homology with ATAD3A with a sequence exemplified by SEQ. ID. NO: 1

As previously mentioned, Alzheimer's disease (AD) is a neurodegenerative disorder characterized by memory loss leading to dementia. It is estimated that in the United States, nearly 6.2 million individuals are impacted by AD dementia, and over 50 million globally. AD is increasing at an alarming pace, projected to double by 2050. According to the World Alzheimer's Report, the total estimated annual worldwide health care costs for persons with AD has reached almost a trillion dollars. Secretory defects in neurotransmitter release and synaptic plasticity, synaptic loss, and mitochondrial dysfunction, have been recognized as early events in AD pathogenesis. Brain regions, especially the entorhinal, temporal and fronto-parietal cortex, the hippocampus and the subcortical nuclei are primarily affected. There is growing evidence that in AD, there is impaired neuronal metabolism due to an increase in free radicals and mitochondrial fission, leading to loss in ATP synthesis required for neurotransmitter release.

As presented herein, the systems level approach in correcting both the metabolic and secretory defects in AD at early stages of the disease, shows great promise. It is important to note that in >90% of Alzheimer's patients, olfactory dysfunction precedes cognitive decline. Moreover, the olfactory bulb of AD mice demonstrate loss in expression of SNAP-25. Since the olfactory bulb is connected via nerves to the brain centers involved in learning, memory and emotion, suggests that the disclosed combinatorial DA1 peptide, either with Apigenin or porosome, would be a viable and highly effective AD therapy, deliverable through the nasal route, especially at the early stages of the disease.

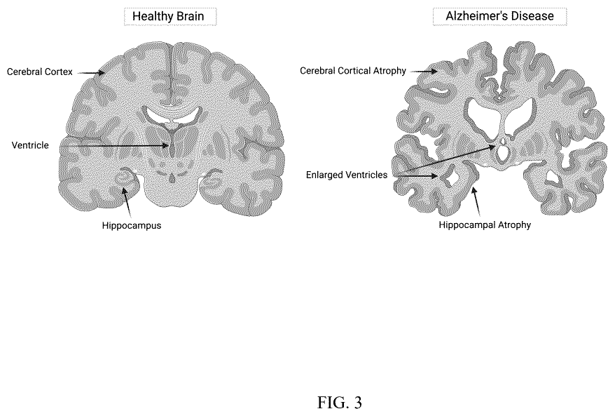

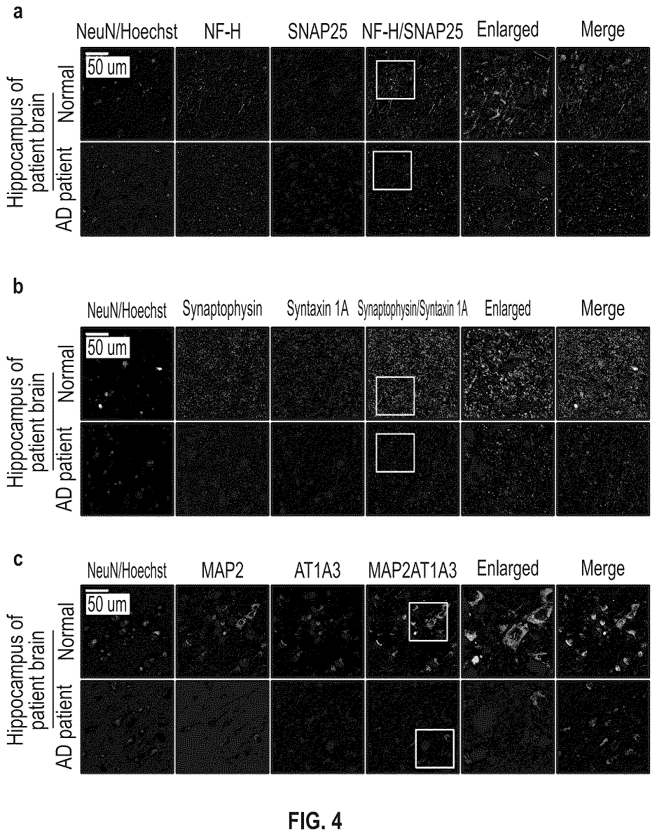

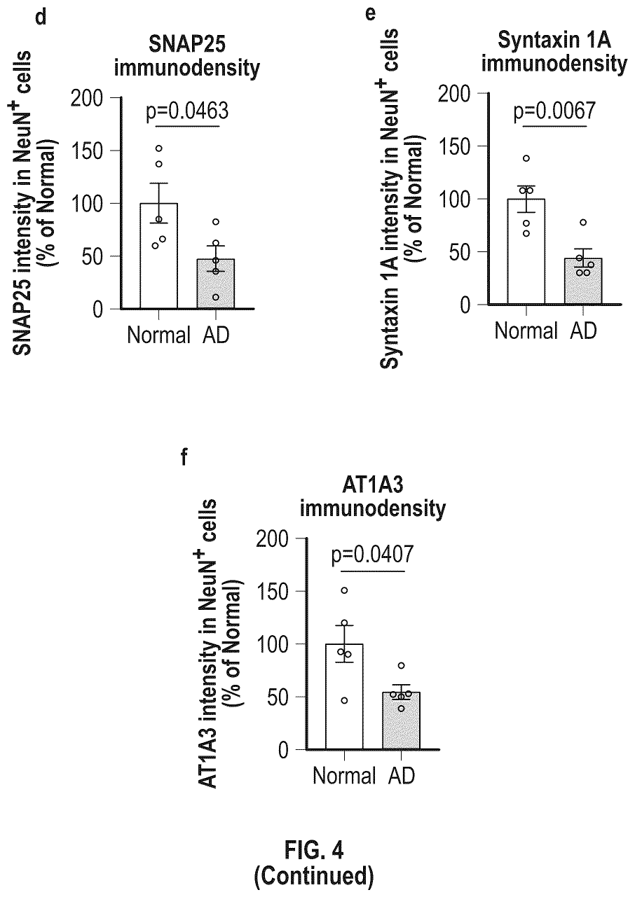

Molecular understanding of the cell's secretory portal, the porosome, and recent progress in artificial intelligence (AI) and the development of AlphaFold™, holds great promise in elucidating the molecular mechanism underlying secretory defects in neurotransmitter release in AD, and a possible pathway to a cure for the disease. Herein demonstrated via immunoblot analysis there is a notable depletion of neuronal porosome proteins SNAP-25, Syntaxin-1A and the sodium/potassium ATPase ATP1A3, is demonstrated in porosomes from AD neurons FIG. 2. Postmortem hippocampus (FIG. 3) sections from control subjects and AD patients, immunostained using SNAP-25-, Syntaxin-1A- and ATP1A3-specific fluorescent antibodies, confirm loss of these porosome proteins in the hippocampus of AD patients (FIG. 4 a-f). These results provided a window to test whether the three porosome proteins SNAP-25, Syntaxin-1A and ATP1A3 could provide superior classification performance in diagnosing AD, and if so, how their interaction within the porosome complex may be negatively impaired and corrected.

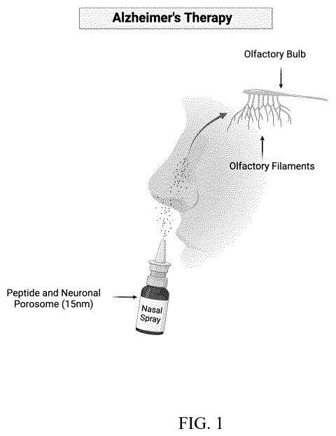

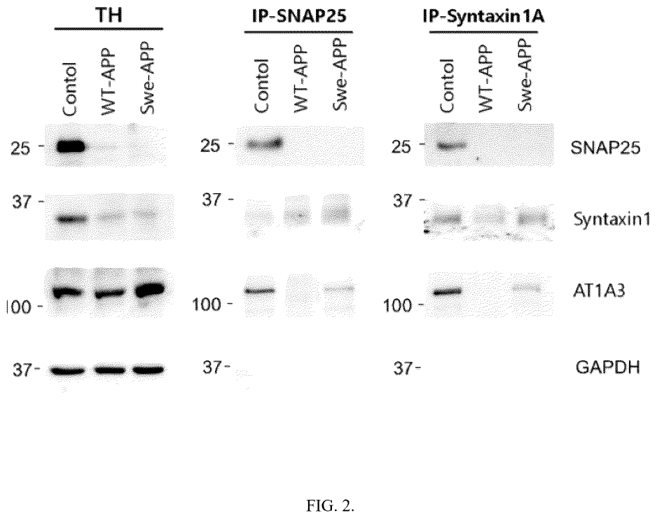

Thus, as seen in an example embodiment in FIG. 1, the methods presented herein permit the development of combinatorial treatments that jointly address the secretory and metabolic effects of AD. As of 2023, there were 187 Phase 1, 2, and 3 clinical trials assessing 141 drugs for AD. Thirty-six drugs were being assessed in Phase 3, 87 in Phase 2, and 31 in Phase 1. Although, neurotransmitter receptors, amyloid plaque, synaptic function, and inflammation were the most common targets of drugs in the pipeline, most therapies have focused on clearing the buildup of amyloid plaques and on reducing inflammation.

In contrast, the subject matter and embodiments herein disclosed take a systems level approach to correcting both the metabolic and secretory defects in AD at early stages of the disease, i.e., hyposmia or olfaction dysfunction. In >90% of Alzheimer's patients, olfactory dysfunction precedes cognitive decline, and the olfactory bulb of AD mice show loss in expression of SNAP-25. Since SNAP-25 levels in the cerebrospinal fluid (CSF) or in blood of AD patients are significantly (p<0.0001) elevated similar to the FDA approved AD biomarker t-tau, further supports SNAP-25 to serve as an AD biomarker. Dysfunction of odor discrimination can therefore also be used as a predictive measure for AD, and the proposed “Odorant Item Specific Olfactory Identification” test where certain odors can differentiate AD from general age-related decline in odor detection could also be used in combination with biomarkers and memory tests, to identify patients for the ‘Metabolic and Secretory Corrector AD Therapy.’

As further seen in FIG. 1, delivery of the proposed therapy is also an important factor. Both the DA1 peptide and Apigenin can pass through the blood-brain barrier. However, since the olfactory bulb is connected via nerves to the brain centers involved in learning, memory and emotion, the DA1 peptide combined either with Apigenin or the porosome, deliverable through the nasal route would be a viable and highly effective AD therapy, deliverable through the nasal route, especially at the early stages of the disease, would be a viable and highly effective AD therapy.

Unique anatomical and physiological features make it very effective to deliver drugs to the brain through the nasal olfactory route, rather than via systemic circulation. Administration of embodiments disclosed herein through the nasal cavity enables drugs to directly reach the brain, bypassing the blood-brain barrier. The nasal route of drug administration to the brain additionally overcomes metabolism of therapeutics which occurs when administered orally. Further advantages include a decrease in the amount of drug required, and minimization of noncompliance issues such as those associated with injectables into systemic circulation. Thus, embodiments of the disclosure favorably provide high pharmacological activity of drugs at lower dosages, and with shorter half-lives (e.g., the administration of drugs that would otherwise decrease in concentration or potency if administered systemically where the drug would be metabolized or removed from circulation).

Embodiments of the disclosure include a pharmaceutically acceptable preparation of apigenin. The preparation may be in a form suitable for administration through the nasal olfactory route. Such administration may be in the form of a suspended mist, powder, solution, nanoemulsion, or other forms such as are known in the art.

There is growing evidence that AD is a consequence of secretory defects in neurotransmitter release that prevent neurotransmission, leading to a loss of neuronal function and cell death in various regions of the brain. Beta-amyloid (AB) peptides are 36 to 43 amino acid long peptides formed as a result of hydrolysis of the amyloid precursor protein (APP), with the 40 and 42 amino acid peptides constitute the main fraction. At nanomolar concentration, Aβ in mammals has been suggested to be an important in facilitating neuronal growth, cellular survival, the modulation of synaptic function and defense against oxidative stress, however at micromolar concentration, the AB 1-42 peptide has been demonstrated to be highly toxic, forming amyloid plaques at later stages of Alzheimer's. In the early stage of the disease, the Aβ 1-42 peptide has been demonstrated to interact with the neuronal porosome protein ATP1A3 and decrease its ATPase activity. Similarly, in a growing number of neurodegenerative diseases, including Alzheimer's, a microtubule-associated protein (MAP) called Tau or MAP-T, has been implicated. Tau protein binds to tubulin also a porosome protein. Binding of Tau to tubulin help form microtubules, essential for a wide range of neuronal function. The porosome protein SNAP-25, has been shown to be a biomarker for neurodegenerative diseases, including Alzheimer's. In Alzheimer's the SNAP-25 protein levels are increased in the cerebrospinal fluid (CSF) as well as in blood samples of patients. The increased level of SNAP-25 correlated with the Tau protein.

Without being bound to a particular theory, embodiments of the disclosure presented herein relate to the specifically impacted porosome protein ATP1A3, a Na+/K+ ATPase by the beta amyloid peptide 1-42. It is demonstrated using in silico (AI) protein-protein interaction studies that the beta amyloid peptide 1-42 disrupts the interactions between ATP1A3, SNAP-25, and Syntaxin-1A and the microtubule associated protein Tau that binds with the porosome protein Tubulin to disrupt porosome function and cause the failure of neurotransmission seen in AD.

Thus, in certain embodiments flavonoids alone, in a carrier, or in a pharmaceutical preparation, are administered through the nasal olfactory route. In certain embodiments, at least one peptide corresponding to a portion of the extracellular domain of the porosome protein ATP1A3 (ATP1A3 peptide), Tau-binding domain of tubulin is also administered. In certain embodiments, the flavonoid is apigenin and the neural disease is Alzheimer's. Targeted dosages may affect secretory function in brain neural cells thus ameliorating the neuronal disorder.

In certain other envisioned embodiments, peptide will bind to the beta amyloid protein 1-42, and prevent it from binding to the porosome protein ATP1A3 and impair porosome function. In certain other embodiments, the ATP1A3-peptide and or Tau-binding domain peptide of tubulin, is administered in conjunction or in a sequence with the flavonoid compound. In still other embodiments, a porosome complex, or parts of a porosome complex is administered. Since the ATP1A3-peptide or Tau-binding domain peptide of tubulin, or the porosome is ‘self’, it is anticipated that there is no immune response or toxicity of this treatment.

Presented in FIG. 2, immunoblot and immunocytochemistry demonstrate depletion of porosome proteins SNAP-25, Syntaxin-1A and Na+/K+-ATPase AT1A3, when neurons are exposed to secreted beta amyloid peptide 1-42 in WT-APP and Swe-APP. Notice the nearly undetectable levels of SNAP-25 protein in the immunoisolated porosome complex. The peptide is known to bind to the extracellular domain of AT1A3 and decrease its ATPase activity. This data shows that the AT1A3 protein interacts with the t-SNARE proteins SNAP-25 and Syntaxin-1A within the neuronal porosome complex.

Presented in FIG. 3 are example diagrams of postmortem hippocampus sections from control subjects and AD patients. Key anatomical structures are and landmarks are provided for reference. Note the atrophy of the cerebral cortex, the enlarged ventricles, and hippocampal atrophy present in the AD affected example when compared to the healthy example.

FIG. 4 with sub-figures FIGS. 4a-c present micrographs of stained postmortem hippocampus sections from control subjects and AD patients. FIGS. 4d-f show quantification of the intensity of SNAP25, Syntaxin 1A, and AT1A3 in NeuN⁺ cells. Postmortem hippocampus sections from control subjects and AD patients (n=5 individuals/group) were stained with antibodies of (FIG. 4 a) anti-SNAP25, anti-Neurofilament heavy chain (NF-H), and anti-NeuN; (FIG. 4 b) anti-Syntaxin 1A, anti-Synaptophysin, and anti-NeuN; (FIG. 4 c) anti-AT1A3, anti-MAP2, and anti-NeuN. The intensity of SNAP25, Syntaxin 1A, and AT1A3 in NeuN⁺ cells was quantified and shown in (FIG. 4 d), (FIG. 4 e) and (FIG. 4 f), respectively. Hoechst was used to label nuclei. All data are presented as mean±SEM. Statistical significance was determined by unpaired Student's t-test.

### AI-Based AD Diagnosis and Therapy

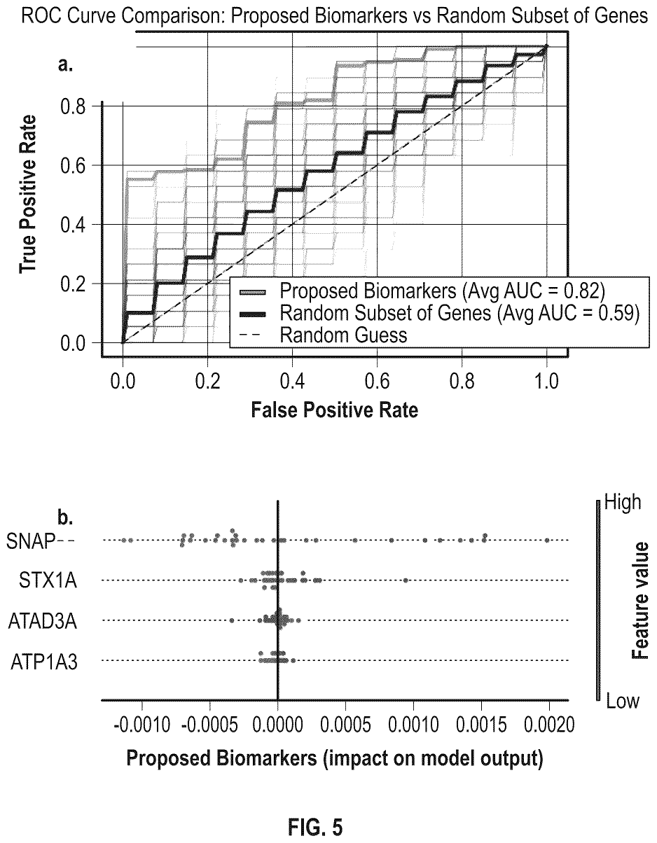

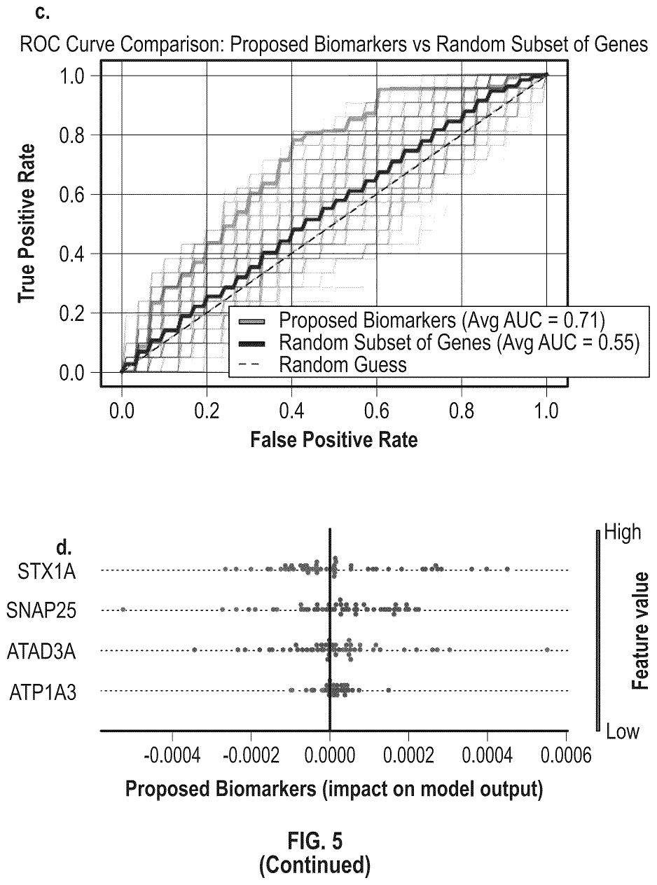

To test whether the three porosome proteins SNAP-25, Syntaxin-1A and ATP1A3 could provide superior classification performance in diagnosing AD, we developed a supervised autoencoder-based neural network that effectively captures complex gene expression patterns while reducing dimensionality. The model consists of an encoder-decoder architecture, where the encoder learns a low-dimensional latent representation of gene expression profiles, preserving the most biologically relevant information, and predicts AD disease status. The model trained on GSE5281 achieved an AUC of 0.82, significantly outperforming random gene subsets AUC of 0.59, demonstrating the improved predictive capacity of the identified biomarkers SNAP-25, Syntaxin-1A and ATP1A3 for Alzheimer's (FIG. 5).

FIG. 5 with sub-figures FIGS. 5a-d presents that porosome proteins SNAP-25, Syntaxin-1A and ATP1A3, and the ATAD3A protein essential for mitochondrial fission and bioenergetics, provide superior classification performance in diagnosing AD over other gene products. Presented in FIG. 5a are Receiver Operating Characteristic (ROC) curves for GSE5281, comparing model performance using selected biomarkers (ATP1A3, ATAD3A, SNAP25, STX1A) against randomly selected gene subsets. The model was run 100 times, selecting a random subset of genes in each iteration. The model trained on GSE5281 achieved an AUC of 0.82, significantly outperforming random gene subsets (AUC=0.59), demonstrating the improved predictive capacity of the identified biomarkers. Presented in FIG. 5b is the SHAP-based biomarker importance for GSE5281. SHAP analysis confirms SNAP25 as the top predictor, with other biomarkers contributing to classification. Each gene's SHAP score is derived from a normalized calculation where the total contribution across all genes sums to 1, providing insight into their relative influence on classification. The color gradient represents feature value distribution, with blue indicating lower expression levels and pink indicating higher expression levels, highlighting the impact of expression variability on model predictions. Presented in FIG. 5c are ROC curves for GSE48350, evaluating classification performance on this dataset. The model was run 100 times, selecting a random subset of genes in each iteration. The targeted biomarkers (ATP1A3, ATAD3A, SNAP25, STX1A) yielded an AUC of 0.71, outperforming randomly selected gene subsets (AUC=0.55), supporting the validity of these markers. Presented in FIG. 5d is the SHAP-based biomarker importance for GSE48350. The color gradient follows the same representation as in FIG. 5b, indicating the relationship between expression levels and classification outcomes.

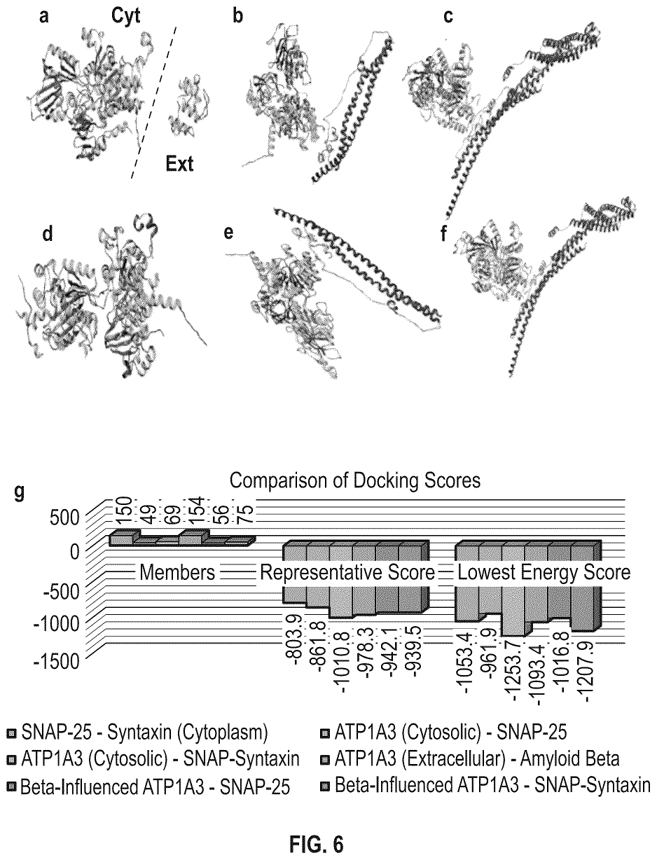

FIG. 6 with sub-figures FIGS. 6a-g illustrates the use of AI to reveal altered binding interactions between porosome proteins SNAP-25, Syntaxin-1 and ATP1A3 when amyloid beta 1-42 peptide binding to the extracellular domain of ATP1A3. This binding of the amyloid beta peptide negatively impacts the binding interactions between ATP1A3-SNAP-25-Syntaxin-1A in the cytosolic domain within the neuronal porosome complex. FIG. 6a presents a structure of ATP1A3, with cytosolic (Cyt.) and extracellular (Ext.) regions separated by a dashed line to indicate their distinct functional compartments. The extracellular region of ATP1A3 is exposed to the extracellular environment, where it interacts with amyloid beta, while the cytosolic region is involved in intracellular interactions with porosome proteins SNAP-25 and Syntaxin-1. Seen in FIG. 6b, the cytosolic domain of ATP1A3 (cyan and purple) favorably binds SNAP-25 (red and green). In FIG. 6c, the interaction between the cytosolic domain of ATP1A3 (cyan and purple) and the SNAP-25+Syntaxin-1A complex (red and green), also favorably interact within the porosome machinery. In FIG. 6d, there is illustrated the binding of amyloid beta (red and green) to the extracellular region of ATP1A3 (cyan and purple). Amyloid beta binds the extracellular region due to its location outside the cell, where it has been implicated in reducing the ATPase activity of ATP1A3 and neurodegeneration. The image includes the entire ATP1A3 protein, with the cytosolic region added for structural context. As seen in FIG. 6e, binding of amyloid beta to the cytosolic domain of ATP1A3 (cyan and purple) and SNAP-25 (red and green), illustrates potential conformational changes in ATP1A3 induced by amyloid beta binding to its extracellular region. In FIG. 6f, the binding of amyloid beta to the cytosolic domain of ATP1A3 (cyan and purple) alters its binding with the SNAP-25+Syntaxin-1A complex (red and green) within the porosome. Finally, as seen in FIG. 6g, there is presented a comparison of docking scores for various interactions involving ATP1A3, SNAP-25, Syntaxin-1A, and amyloid beta (1-42) peptide. Results show that binding of amyloid beta (1-42) peptide to the extracellular domain of ATP1A3, results in tighter binding (lower energy scores) with SNAP-25 and reduced binding (higher energy scores) with the SNAP-25-Stntaxin-1 complex. This suggests that amyloid beta binding to the extracellular region of ATP1A3 may induce structural changes that enhance intracellular interactions with SNAP-25. These results suggest that while amyloid-beta binding enhances ATP1A3-SNAP-25 interactions, it negatively impacts the formation or stability of the larger ATP1A3-SNAP-25-Syntaxin-1A complex within the neuronal porosome, resulting in impaired secretion of neurotransmitters.

These results suggest that while Aβ (1-42) binding enhances ATP1A3-SNAP-25 interactions, it negatively impacts the stability of the larger ATP1A3-SNAP-25-Syntaxin-1A complex within the neuronal porosome, resulting in impaired secretion of neurotransmitters. This altered binding negatively impacting the porosome may also result in the loss of these porosome proteins, as demonstrated, resulting in neurodegeneration and progression of AD. Understanding the molecular interacting domain of ATP1A3 where the AB (1-42) binds, provides for the first time, a molecular-level understanding of the role played by the AB (1-42) peptide in impaired neurosecretion in Alzheimer's, allowing the development of engineered peptide biologics, those that could appropriately fold and bind with greater affinity with the AB (1-42) peptide and neutralize it, serving as “decoy peptides” in treating AD. A “decoy peptide” in this sense is an engineered peptide biologic designed to target and block a binding and/or interaction motif of a target protein.

For example, in the case of two proteins known to interact and/or bind with each other there are specific regions on the first and second proteins that facilitate the interaction and/or binding. A “decoy peptide” can be designed to mimic a binding/interaction motif of a first protein in order to more tightly bind/interact with the second protein. The net effect is an artificial inducement of a decrease or prevention of interaction/binding of the two proteins. In the context of AD, as above described, plaque formation may be impaired or blocked. This approach may be extended to additional secretory diseases such as those in the below examples and those known to be affected by porosome complex irregularities.

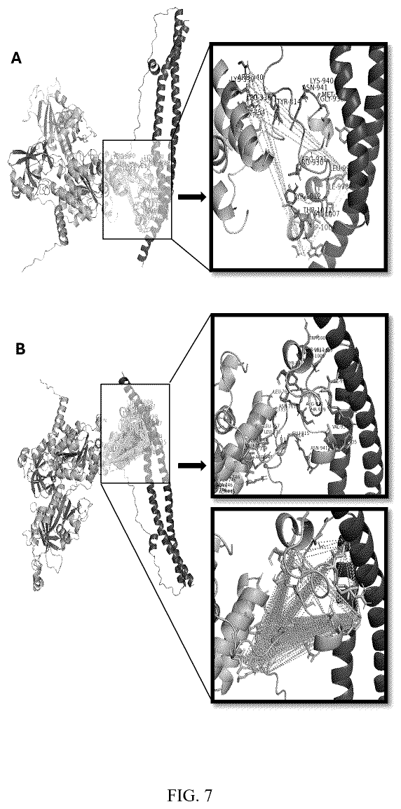

FIG. 7 with sub-figures FIGS. 7A and 7B, presents high-resolution images of the detailed molecular interaction between ATP1A3 and SNAP 25, with and without Amyloid-β binding. FIG. 7 illustrates the structural integration of ATP1A3 and SNAP25, highlighting residues within 5 Å of the ligand. FIG. 7A, ATP1A3-SNAP25 Interaction: The interaction without Amyloid-β influence involves 19 key residues (LEU 336, LYS 339, ARG 340, GLU 753, GLU 754, TYR 814, ARG 930, ARG 931, GLY 938, MET 939, LYS 940, ASN 941, LEU 997, ILE 998, GLY 1005, TRP 1006, VAL 1007, THR 1011, TYR 1012). FIG. 7B, ATP1A3-SNAP25 Interaction in the presence of Amyloid-β: With Amyloid-β influence, the number of interacting residues increases to 28, including additional residues such as CYS 333, LEU 757, ILE 758, ASN 761, LEU 762, GLU 815, THR 929, VAL 934, PHE 935, TYR 1013. A notable difference in (B) Amyloid-β-influenced ATP1A3-SNAP25 binding is the increase in hydrogen bonds (yellow dashed lines), which were identified based on a 1.8 Å distance threshold. This suggests stronger and potentially altered interactions in the presence of the Amyloid-β peptide. Hydrophilic (skyblue) and hydrophobic (orange) residues are also highlighted, to provide a clearer view, a zoomed-in version of the key residues and hydrogen bonding interactions.

Since amyloid beta (AB) has been found to alter the connectivity of olfactory neurons in the absence of amyloid plaques, and SNAP25 is a potential target for early stage Alzheimer's disease, the binding of AB (1-42) to the extracellular domain of the ATP1A3 protein, and its impact on the cytosolic facing domain of ATP1A3 with SNAP-25, and Syntaxin-1A interactions within the neuronal porosome complex was assessed using AI. This was examined using a multi-step computational workflow combining structure prediction, molecular docking, and structural analysis. This study revealed that binding interactions between porosome proteins SNAP-25, Syntaxin-1 and ATP1A3 exist, which is impaired upon binding of Aβ (1-42) to the extracellular domain of ATP1A3 (FIGS. 6, 7). Results show that binding of Aβ (1-42) peptide to the extracellular domain of ATP1A3, results in tighter binding (lower energy scores) with SNAP-25 and reduced binding (higher energy scores) with the SNAP-25-Syntaxin-1 complex. These results suggest that while AB (1-42) binding enhances ATP1A3-SNAP-25 interactions, it negatively impacts the stability of the larger ATP1A3-SNAP-25-Syntaxin-1A complex within the neuronal porosome, resulting in impaired secretion of neurotransmitters.

This altered binding negatively impacting the porosome, may also result in the loss of these porosome proteins as shown in FIG. 2, resulting in neurodegeneration and progression of AD. These results, also provide for the first time, a molecular understanding of how the Aβ (1-42) peptide negatively impacts neurosecretion, providing valuable insights into the treatment of AD. Since multiple proteins within the neuronal porosome complex are negatively impacted and some proteins are lost from the complex, this suggests that introduction of normal functioning porosomes containing a complete assembly of proteins into AD neurons would be the most effective therapeutic approach compared to compensating individual porosome proteins via overexpression or reconstitution into AD neurons. Furthermore, the structure of the molecular interacting domain of ATP1A3 where the AB (1-42) binds, also provides a window for the development of engineered peptide biologics, those that could appropriately fold and bind with greater affinity with the Aβ (1-42) peptide and neutralize it.

### Secretory and Metabolic Correctors for AD Therapy

Porosomes are secretory portals at the cell plasma membrane where secretory vesicles transiently dock and fuse to expel a precise amount of intra-vesicular contents from the cell during secretion. In neurons, porosomes are 15 nm cup-shaped lipoprotein structures at the presynaptic membrane, composed of nearly 30 proteins. Several porosome proteins have previously been implicated in neurotransmission and neurological disorders, attesting to the crosstalk between porosome proteins and their coordinated involvement in release of neurotransmitter at the synapse. In Alzheimer's, levels of porosome proteins CNPase (2,3-cyclic nucleotide phosphodiesterase) and the heat shock protein 70 (HSP70) are found to increase, while the levels of dihydropyrimidinase-related protein-2 (DRP-2) decrease. Decreased levels of CNPase have been observed in the frontal and temporal cortex of patients with AD. Similarly, porosome proteins SNAP-25 and synaptophysin are significantly reduced in neurons of patients with Alzheimer's disease. Mice that are SNAP-25 (+/−) show disabled learning and memory, and exhibit epileptic like seizures. These results support the conclusion that, alteration of one porosome protein impacts others within the complex, resulting in impaired porosome-mediated secretion. This finding is similar to recent studies on human bronchial epithelial (HBE) cells which showed that the AF508 cystic fibrosis transmembrane conductance regulator (CFTR) mutation in HBE cells, affects nearly a dozen porosome proteins, including CFTR, within the porosome complex. Therefore, the reprogramming of the porosome secretory machinery into the cell plasma membrane of Cystic Fibrosis (CF) cells, was able to rescue from CF. Similarly, we hypothesized that reconstitution of the 15 nm normal neuronal porosome complex in Alzheimer's neurons would overcome secretory defects in neurotransmitter release.

Similarly, it is widely accepted that the mitochondrial production of reactive oxygen species (ROS) contributes to the detrimental alterations in the etiology and/or progression of many pathological conditions, including brain neurodegeneration. Studies report that flavonoids can protect cells from different insults that lead to mitochondria-mediated cell death, and epidemiological data further show that some of these compounds attenuate the progression of diseases associated with oxidative stress and mitochondrial dysfunction. Flavonoids are low molecular weight phenolic compounds, displaying significant ROS scavenging capability, including other cellular antioxidant effects, and are hence ideal for the protection of brain neurons from the buildup of free radicals generated by the mitochondria leading to mitochondrial fission in Alzheimer's.

We developed DA1, a synthetic peptide that binds to ATAD3A and reduces ATAD3A oligomerization, which in turn decreases Drp1/ATAD3A binding that occurs during AD progression. DA1 treatment reduces mitochondrial bioenergetic defects, and improves the survival rate of neurons exposed to toxic AB in vitro. DA1 penetrates the blood-brain-barrier and is nontoxic with minimal effects on the immune response. These physiological and pharmacodynamic properties make DA1 a promising candidate for AD therapy. We tested the in vivo efficacy of DA1 in 5×FAD AD transgenic mice using a subcutaneous (SQ) Alzet mini-pump, delivering either the control peptide TAT or DA1 starting at 6 weeks of age show that 5×FAD mice treated with the control peptide exhibited enhanced ATAD3A oligomerization at 6 months of age, which was abolished by treatment with DA1. Immunohistochemistry of 6-month-old 5×FAD mice brain revealed a significant increase in the density and area covered by AB. DA1 treatment significantly reduced this amyloid load. Furthermore, DA1 decreased immunoreactivity of GFAP (astrocytes) and Iba1 (microglia), suggesting reduced neuroinflammation. Additionally, DA1 treatment from 2 to 8 months of age improved the long-term memory of 5×FAD mice. DA1 treatment had no effect on the behavior of WT mice, further supporting the nontoxic nature of the peptide. We further developed the linear DA1 peptide into a cyclic DA1 analogue that has improved stability and efficacy.

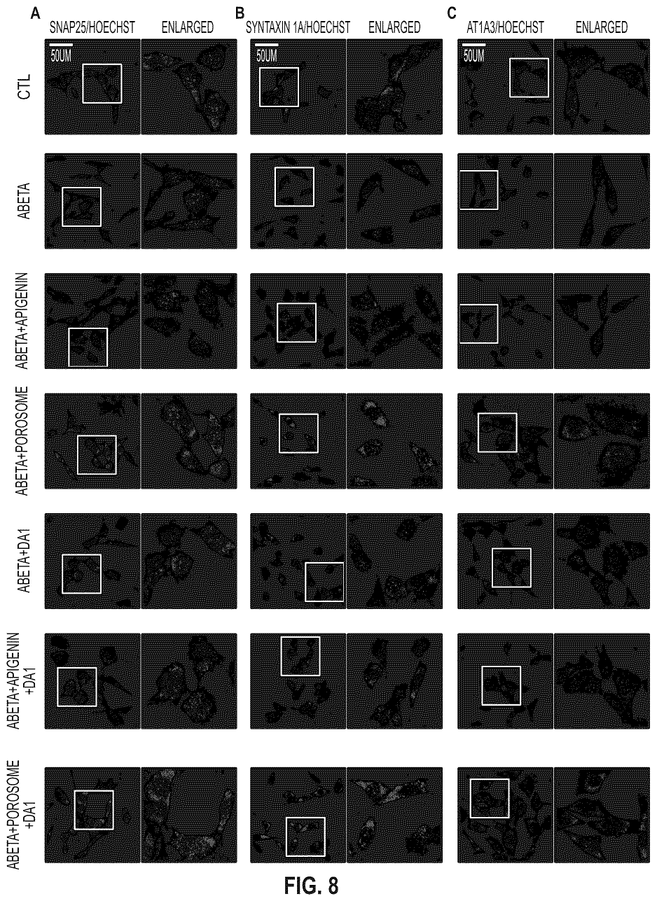

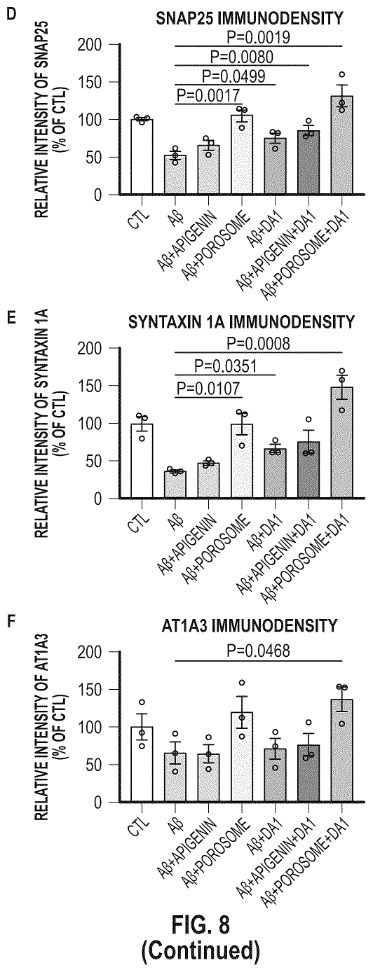

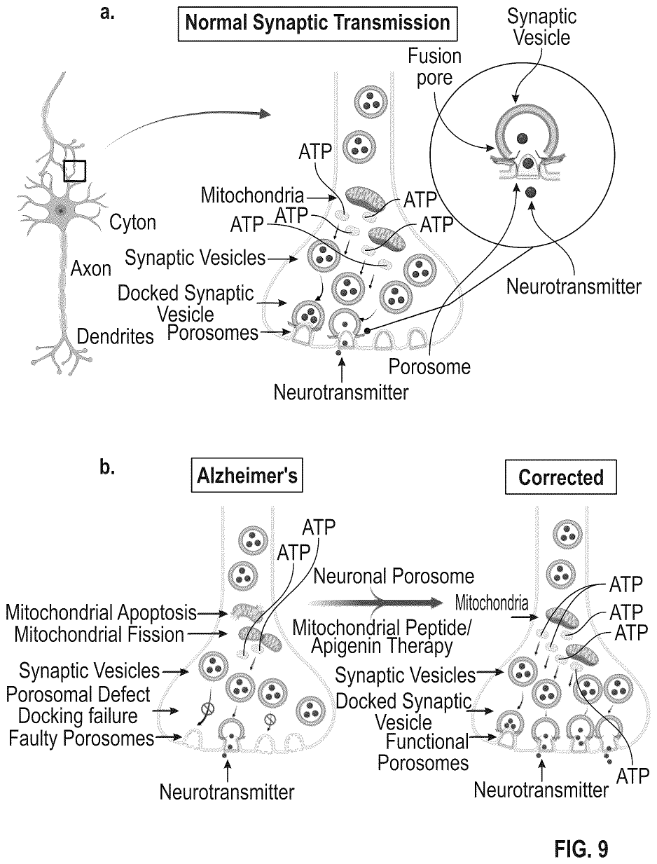

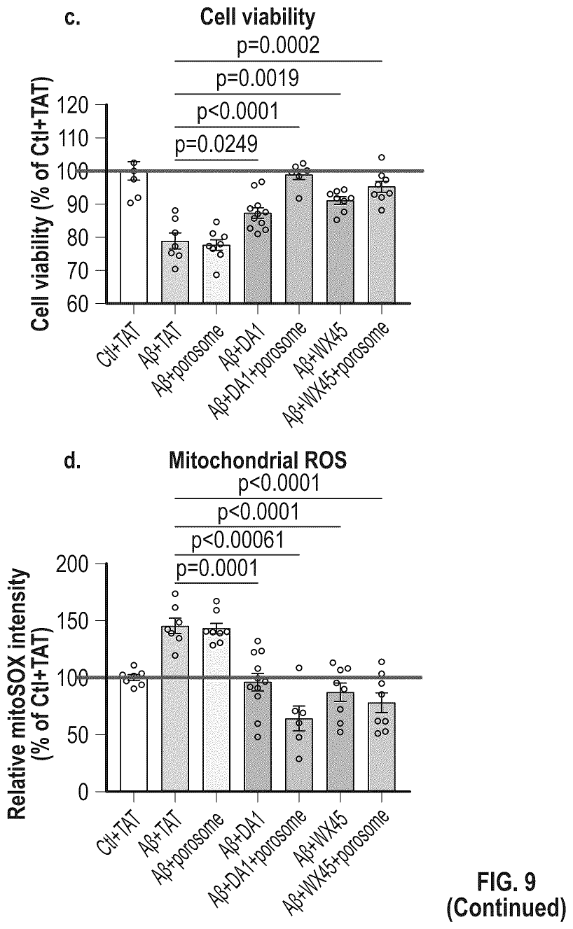

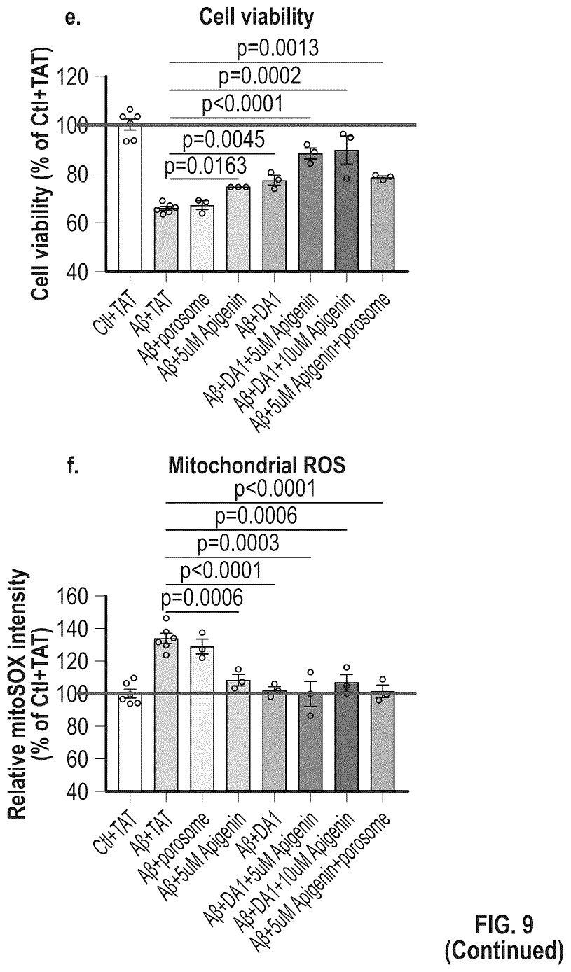

In the present disclosure in AD neurons, both the linear and circular DA1 peptide (WX45) similarly demonstrated a partial rescue from AD both in cell viability and in reduced mitochondrial superoxidase activity (mitoSOX) in Alzheimer's neurons (Aβ+TAT) (FIG. 9). The WX45 circular DA1 peptide demonstrated to be more effective than the linear DA1 peptide. However as hypothesized, the neuronal porosome reconstitution combined with the DA1 peptide especially the circular WX45 peptide inhibitor of ATAD3A oligomers, was able to restore AD neurons to near normal levels of viability and greatly reduced oxidative stress in mitochondria, demonstrating the potential for therapeutic applications. Additionally, the peptide and apigenin, a small molecule flavonoid considered safe by the food and drug administration (FDA) and used as a food additive, were also able to greatly increase viability in AD neurons and reduce to near normal levels mitoSOX (FIG. 9), also demonstrating its use in AD therapy. Collectively, these results demonstrate great promise in the combinatorial use of the porosome reconstitution secretory reprogramming and the DA1 peptide and Apigenin metabolic correctors, as highly safe and effective AD therapies. In agreement, our immunoblot analysis and immunocytochemistry study of human brain tissue (FIGS. 2 and 4), and the mouse neuroblastoma Neuro2a control cells, and our APPwt and sweAPP Alzheimer's cells (FIG. 8), demonstrate significant decrease in SNAP-25 immunoreactivity and altered presence of other porosome proteins in both APPwt and sweAPP neurons, confirming earlier reported studies of a decrease in SNAP-25 in patients with Alzheimer's. Consequently, in Alzheimer's patients there is an increase in levels of SNAP-25 in the cerebrospinal fluid, which has been associated with cognitive decline. Upon porosome reconstitution in APP neurons, our immunocytochemistry demonstrates the restoration of SNAP-25 and other porosome-associated proteins (FIG. 8) that result in increased cell viability to near normal levels and a decrease in mitoSOX activity (FIG. 9). In summary, we have tested the combinatorial use of the porosome, the small flavonoid molecule Apigenin, and our linear and cyclic DA1 peptides, in treating AD. Results from our study demonstrate great promise in the combinatorial use of secretory (porosome) and metabolic (flavonoid and DA1 peptide) correctors for AD therapy, restoring AD neurons to near normal levels of viability and greatly reducing oxidative stress in the mitochondria.

FIG. 8 with sub-figures FIGS. 8a-f, presents the immunocytochemistry demonstrating depletion of porosome proteins SNAP-25, Syntaxin-1A, and Na⁺/K⁺ Transporting ATPase alpha 3 (AT1A3) in Alzheimer's neurons WT-APP (Aβ), which is fully restored to normal levels following porosome-reconstitution therapy, and partially by Apigenin and the DA1 peptide. The combined exposure to both the porosome and DA1 peptide, further enhances the expression of all three porosome proteins SNAP-25, Syntaxin-1A, and AT1A3. Modest increase in SNAP-25, Syntaxin-1A, or AT1A3 immunoreactivity is observed following exposure of Apigenin to the Alzheimer's neurons (+Aβ). Note that Apigenin and DA1 combination has a significant effect on enhancing expression of all three porosome proteins. FIG. 8a: In this panel, the immunolocalization of SNAP-25 in untreated control neurons (Ctl), Aβ (Alzheimer's) neurons, Aβ neurons treated with Apigenin (Aβ+Apigenin), Aβ neurons reconstituted with neuronal porosomes, Aβ treated with the DA1 peptide, Aβ treated with Apigenin and the DA1 peptide, and Aβ treated with porosome and the DA1 peptide, is shown. FIG. 8b: In this panel, the immunolocalization of Syntaxin-1A in untreated control neurons (Ctl), Aβ (Alzheimer's) neurons, Aβ neurons treated with Apigenin (Aβ+Apigenin), Aβ neurons reconstituted with neuronal porosomes, Aβ treated with the DA1 peptide, Aβ treated with Apigenin and the DA1 peptide, and Aβ treated with porosome and the DA1 peptide, is shown. FIG. 8c: In this panel, the immunolocalization of AT1A3 in untreated control neurons (Ctl), Aβ (Alzheimer's) neurons, Aβ neurons treated with Apigenin (Aβ+Apigenin), Aβ neurons reconstituted with neuronal porosomes, Aβ treated with the DA1 peptide, Aβ treated with Apigenin and the DA1 peptide, and Aβ treated with porosome and the DA1 peptide, is shown. FIG. 8d: In this bar graph, the quantification of the immunolocalization of SNAP-25 in untreated control neurons (Ctl), Aβ (Alzheimer's) neurons, Aβ neurons treated with Apigenin (Aβ+Apigenin), Aβ neurons reconstituted with neuronal porosomes, Aβ treated with the DA1 peptide, Aβ treated with Apigenin and the DA1 peptide, and Aβ treated with porosome and the DA1 peptide, is shown FIG. 8e: In this bar graph, the quantification of the immunolocalization of Syntaxin-1A in untreated control neurons (Ctl), Aβ (Alzheimer's) neurons, Aβ neurons treated with Apigenin (Abβ+Apigenin), Aβ neurons reconstituted with neuronal porosomes, Aβ treated with the DA1 peptide, Aβ treated with Apigenin and the DA1 peptide, and Aβ treated with porosome and the DA1 peptide, is shown. FIG. 8f: In this bar graph, the quantification of the immunolocalization of AT1A3 in untreated control neurons (Ctl), Aβ (Alzheimer's) neurons, Aβ neurons treated with Apigenin (Aβ+Apigenin), Aβ neurons reconstituted with neuronal porosomes, Aβ treated with the DA1 peptide, Aβ treated with Apigenin and the DA1 peptide, and Aβ treated with porosome and the DA1 peptide, is shown.

FIG. 9 with sub-figures FIGS. 9a-f, presents the combinatorial therapy of porosome reconstitution the secretory corrector, and the DA1 peptide inhibitor of mitochondrial fission, the metabolic corrector, restores cell viability and reduces oxidative stress in mitochondria to normal levels in AD neurons. Similarly, combination therapy of the small flavonoid molecule Apigenin and the mitochondrial fission inhibiting linear DA1 peptide results in increase cell viability and reduction of mitochondrial oxidative stress to near normal levels in AD neurons. FIG. 9a: Schematic illustration of a synapse at the nerve ending in healthy neurons, demonstrating normal mitochondrial function, energy (ATP) generation, and porosome-mediated neurosecretion. FIG. 9b: Schematic illustration of a synapse at the nerve ending in AD neurons, demonstrating abnormal mitochondrial function resulting in mitochondrial fission, loss in ATP generation, and altered porosome-mediated neurosecretion. All of these defects in AD are seen to be overcome using porosome reconstitution and or the DA1 peptide inhibitors of mitochondrial fission therapy. FIG. 9c: Cell viability is significantly enhanced in Alzheimer's neurons (Aβ+TAT) by porosome reconstitution either in the presence of the linear or the circular DA1 peptide WX45. HT-22 cells were pretreated with porosome (175 ng per well in a 96-well plate) overnight, followed by treatment with oligomeric Aβ1-42 peptides (5 μM) together with DA1 (linear peptide) or WX45 (DA1 cyclic peptide) (1 μM) for 40 h. Note that the cell viability in Alzheimer's neurons following porosome reconstitution and the presence of the circular peptide WX45, demonstrate recovery to healthy neuron levels. Cell viability was measured by MTT assay after 16 h of serum starvation. Data represent the mean±SEM from at least six independent experiments. Statistical significance was determined by one-way ANOVA with Tukey's post hoc test. ** p<0.01; *** p<0.001. **** p<0.0001. FIG. 9d: Porosome reconstitution significantly reduces mitochondrial superoxidase activity (mitoSOX) in Alzheimer's neurons (Aβ+TAT) in presence of either the linear DA1 or circular DA1 (WX45) peptide. HT-22 cells were pretreated with porosome (175 ng/well in a 96-well plate) overnight. Cells were then pre-incubated with DA1 or WX45 DA1 cyclic peptide (1 μM) for 1 hour, followed by treatment with oligomeric Aβ₁₋₄₂ peptides (10 μM) for 12 hours. Mitochondrial superoxide production was assessed 1 hour after the second peptide treatment using MitoSOX staining. MitoSOX activity in Alzheimer's neurons following porosome reconstitution demonstrate recovery to much reduced levels than found in healthy neurons in the presence of the circular peptide WX45. FIG. 9e: Viability of mouse hippocampal HT-22 neuronal cells exposed to 5 μM toxic oligomeric Aβ₁₋₄₂ peptides mimicking Alzheimer's (Aβ+TAT), is significantly enhanced when treated with 1 μM DA1 linear peptide and 5 μM or 10 μM Apigenin. Cells were treated with DA1 1 μM+Aβ 5 μM+Apigenin (5 μM or 10 μM). After 24 h, cells were washed using serum-free media, and again treated with peptides 1 μM+Aβ 5 μM+Apigenin (5 μM or 10 μM) in serum-free medium. Cell viability was measured by MTT assay at 40 h (16 h serum starved). Data represent the mean±SEM from at least six independent experiments. FIG. 9f: Mitochondrial superoxidase activity (mitoSOX) in Alzheimer's neurons (Aβ+TAT) is significantly reduced in the presence of the linear DA1 peptide. However, not much change is observed in the presence of either 5 μM or 10 μM of Apigenin. Mouse hippocampal HT-22 neuronal cells exposed to DA1 1 μM+Apigenin (5 μM or 10 μM) for 1 h and 10 μM Aβ (oligomeric Aβ₁₋₄₂ peptides) for 12 h. Cells were again treated with DA1 1 μM+Apigenin (5 μM or 10 μM) again 1 h before staining for mitoSOX (measure mitochondrial ROS). Data represent the mean±SEM from at least six independent experiments. Statistical significance was determined by one-way ANOVA with Tukey's post hoc test. * p<0.05; ** p<0.01.

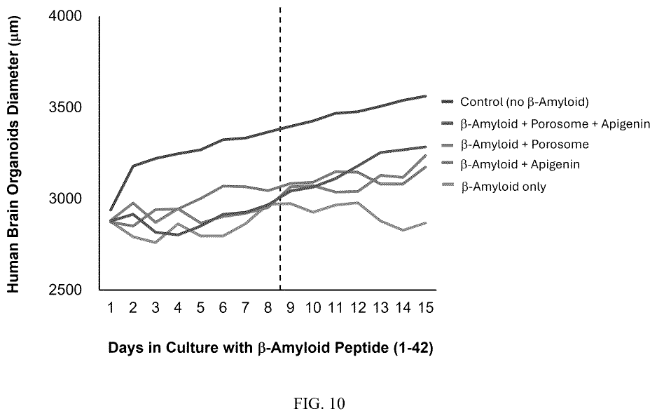

FIG. 10 illustrates porosome reconstitution and apigenin treatment overcomes the negative impact of beta amyloid (1-42) on the growth of human brain organoids in culture. There is over a 50% rescue following 15 days after porosome treatment. Human iPSC-derived cerebral organoids were purchased from Acro Biosystems. (Cat No. CIPO-BWL001K, DE. USA). Organoids were seeded to a 96-well ultra-low attachment plate with one organoid into the well with 200 μL of cerebral organoid maintenance and differentiation medium per well (Acro Biosystem, Cat No. RIPO-BWM003). The plate was on an orbital shaker with a speed of 100 rpm at 37° C., 5% CO₂. The media were changed every other day. After 30 days, the organoids were cultured with beta-Amyloid (10 μM) to induce Alzheimer's disease condition. 7 days after adding beta-Amyloid (Abcam, Cat No. AB120301, Cambridge. UK). Pososome (175 ng/well), Apigenin (10 μM, Selleckchem, Cat. No S2262, TX USA) were treated separately or together with beta-Amyloid for 1 day. After that, half of the medium of well (100 μL) was replaced with new medium including beta-Amyloid every day. As seen in FIG. 10 control organoids with no beta amyloid exposure attained the greatest diameter. Organoids exposed only to beta amyloid maintained the lowest diameter and exhibited almost no change in size across the study. Finaly, treated organoids showed increased growth above the beta-amyloid exposed organoids. This data is further summarized and presented in Table 1 which further details how the complete loss of human brain organoid growth in the presence of beta amyloid peptide (1-42) is reversed by over 50% within 15 days following one treatment of prosomes and/or apigenin.

TABLE 1
Size dynamics of human brain organoids exposed to beta amyloid and treated.
Dimeter of organoids (μm, Mean ± S.E.M)

| | | β-Amyloid | | | |
|---|---|---|---|---|---|
| Day | No β-Amyloid | No treatment | Porosome | Apigenin | Porosome + Apigenin |
| 1 | 2937.7 ± 158.0 | 2874.7 ± 213.4 | 2881.2 ± 56.8 | 2871.8 ± 138.2 | 2877.4 ± 48.8 |
| 2 | 3179.1 ± 102.0 | 2791.7 ± 226.7 | 2970.2 ± 21.1 | 2850.3 ± 192.7 | 2914.0 ± 66.5 |
| 3 | 3220.3 ± 65.0 | 2760.2 ± 236.4 | 2870.8 ± 29.6 | 2940.0 ± 357.0 | 2816.3 ± 42.7 |
| 4 | 3246.8 ± 58.2 | 2862.8 ± 289.6 | 2944.5 ± 112.8 | 2943.8 ± 287.2 | 2801.1 ± 19.1 |
| 5 | 3287.4 ± 49.2 | 2796.7 ± 310.8 | 2867.1 ± 79.3 | 3001.0 ± 290.3 | 2849.9 ± 84.6 |
| 6 | 3323.3 ± 28.1 | 2796.7 ± 379.9 | 2902.3 ± 80.8 | 3089.2 ± 299.8 | 2914.0 ± 15.3 |
| 7 | 3332.1 ± 37.6 | 2862.8 ± 356.4 | 2919.0 ± 36.2 | 3065.6 ± 352.9 | 2926.2 ± 50.2 |
| 8 | 3364.5 ± 49.2 | 2969.4 ± 334.9 | 2961.6 ± 124.9 | 3044.1 ± 377.7 | 2986.8 ± 69.1 |
| 9 | 3396.9 ± 47.1 | 2973.4 ± 312.6 | 3064.1 ± 180.1 | 3083.8 ± 366.7 | 3042.1 ± 76.2 |
| 10 | 3426.3 ± 61.0 | 2926.0 ± 318.3 | 3071.2 ± 160.9 | 3099.8 ± 369.4 | 3063.5 ± 74.1 |
| 11 | 3467.5 ± 32.4 | 2965.5 ± 319.4 | 3036.0 ± 113.2 | 3148.2 ± 387.3 | 3109.3 ± 98.2 |
| 12 | 3476.4 ± 21.2 | 2977.3 ± 347.0 | 3039.5 ± 80.0 | 3144.6 ± 401.4 | 3179.4 ± 101.2 |
| 13 | 3505.8 ± 45.7 | 2878.6 ± 382.4 | 3127.5 ± 33.8 | 3080.0 ± 345.5 | 3252.7 ± 92.3 |
| 14 | 3538.2 ± 51.3 | 2827.3 ± 399.6 | 3116.9 ± 33.6 | 3080.0 ± 347.1 | 3267.9 ± 85.1 |
| 15 | 3567.7 ± 63.7 | 2866.8 ± 378.6 | 3236.6 ± 70.4 | 3173.3 ± 293.6 | 3283.2 ± 69.0 |

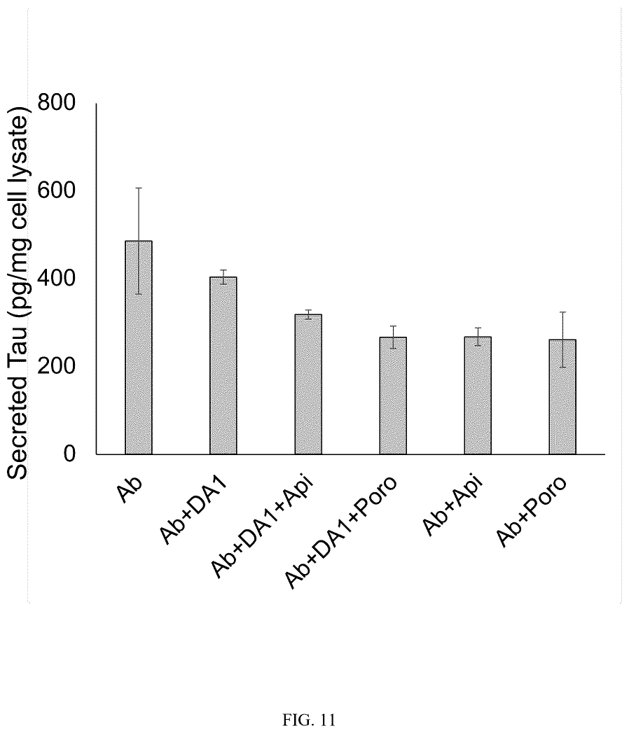

In a combinatorial strategy of reprogramming the neuronal secretory and metabolic components, we established a dual-target therapeutic framework that repairs synaptic and mitochondrial defects to counteract neurodegeneration in Alzheimer's. Since Tau pathology follows the abnormal accumulation of Aβ plaques, we tested the release of Tau from AD neurons in culture following our dual targeted therapy. Interestingly, the greatest drop in Tau release from AD neurons followed porosome and Apigenin treatment FIG. 11 illustrates that a treatment of Alzheimer's mouse hippocampal HT-22 neuronal cells (Aβ) with porosome reconstitution decreases Tau release. HT-22 cells exposed to 5 μM toxic oligomeric Aβ1-42 (Ab) peptides mimicking Alzheimer's Disease (AD). HT-22 AD neuronal cells were treated with porosome (Poro, 175 ng per well in a 96-well plate), Apigenin (Api, 10 μM), and DA1 (1 μM) either alone or in combination. Note porosome and apigenin treatment shows the greatest reduction of Tau secretion from neurons. Tau measured using ELISA.

As envisioned, therapies based on embodiments disclosed herein are scalable for commercialization since the DA1 circular peptide and Apigenin are readily available, and the scale-up of the neuronal porosome biologic could be achieved by immunoisolation from 3D cultures of the human neuroblastoma SH-SY5Y cell line grown in bioreactors. 3D neuronal cultures will be used since the capacity of a neuron to establish synaptic connections is an essential prerequisite for its functional maturation, and that the formation of the synapse involves the appropriate assembly of the porosome secretory machinery at the nerve terminal (FIG. 9a). Since the neuronal porosome is present in all neurons, the above-mentioned scale-up approach offers reproducibility, reliability and safety. One needs to be critically aware that, since three (FIG. 8) among possibly others, of the nearly 30 or so proteins of the neuronal porosome complex are impacted in Alzheimer's, resulting in impairment function, one safe and highly effective approach would be to ameliorate the impairment by introducing normal neuronal porosomes at the pre-synaptic membrane. Porosome reconstitution therapy therefore remains the most optimal approach in ameliorating neurotransmitter release defects in AD. The normal secretion of neurotransmitter and its timely breakdown is physiologically relevant and optimal, as opposed to the available AD medications such as cholinesterase inhibitors that prevent the breakdown of acetylcholine in the brain to improve neuronal communication. Cholinesterase inhibitor drugs exhibit side effects that may include nausea, vomiting, diarrhea, muscle cramps, fatigue and even weight loss.

### EXAMPLES

### Example 1

In an envisioned embodiment, a composition comprising a synthetic blood-brain barrier traversing peptide mimicking a specific domain of ATP1A3 and or Tubulin, either alone or in combination with porosome reconstitution and optionally including the flavonoid compound Apigenin is administered through the nasal olfactory route for the purposes of treating a neurodegenerative disease (e.g., Alzheimer's Disease).

### Example 2

In an alternative to the embodiment of Example 1, a pharmaceutical preparation may be prepared for administration either directly to the neural tissue (e.g., via an injection into the interior of the cranium or spinal canal) or in concert with one or more so-called blood-brain-barrier shuttle molecules.

The disclosed compositions and methods offer promising new applications beyond Alzheimer's Disease and related neurodegenerative disorders.

### Example 3

In an envisioned embodiment, the combination of porosome complex, DA1 peptide, and apigenin may be administered via the nasal olfactory route to patients following acute traumatic brain injury (TBI), with the goal of restoring neuronal secretory function, reducing oxidative stress, and promoting neuroregeneration. Similarly, this composition could be utilized to ameliorate chemotherapy-induced cognitive impairment, commonly known as “chemo brain,” by targeting mitochondrial dysfunction and synaptic loss in neural tissue of cancer patients post-chemotherapy.

### Example 4

In another envisioned embodiment, in elderly patients experiencing age-related olfactory dysfunction the composition may be applied as a nasal spray to restore olfactory neuron function and potentially prevent progression to neurodegenerative disease.

### Example 5

In another envisioned embodiment, as an adjunctive treatment for refractory epilepsy, co-administration of the porosome complex and DA1 peptide may help stabilize synaptic transmission and reduce seizure frequency by correcting secretory and metabolic defects in neurons. The composition also holds potential for use in autism spectrum disorder (ASD), where modulation of synaptic function and neurotransmitter release could improve behavioral and cognitive outcomes.

### Example 6

In an additional envisioned embodiment, for patients with diabetes-induced peripheral neuropathy, administration of the composition may restore neuronal metabolic function and reduce neuroinflammation.

### Example 7

In the context of stroke recovery, the composition could be applied post-stroke to promote neuronal survival, enhance synaptic repair, and reduce oxidative damage in affected brain regions.

### Example 8

Additionally, the composition may be utilized to target secretory and metabolic dysfunctions in multiple system atrophy (MSA), aiming to slow disease progression and improve motor and autonomic symptoms.

These expanded applications illustrate the versatility and therapeutic potential of the disclosed technology across a broad spectrum of neurological and neurodegenerative conditions.

### Materials and Methods

Porosome Isolated from Neuro2a Wt (Control) for Western Blot Analysis and for Reconstitution into Alzheimer's (Experimental) Neuro2a APPwt, and Neuro2a APPswe Cells

Neuro 2a wt cells were used to isolate porosomes for reconstitution. RIPA buffer (10 mM Tris-HCl, pH 8.0·1 mM EDTA·0.5 mM EGTA·1% Triton X-100·0.1% Sodium Deoxycholate·0.1% SDS·140 mM NaCl. Dilute with dH₂O) containing 0.1 mM PMSF, 1 mM ATP, and a cocktail of protease inhibitors, was used to lyse the cells. Protein in all fractions was estimated using BCA Protein assay Kit (ThermoFisher Cat. No. 23227, Rockford, IL 61101, USA). A total of 500 μg of the supernatant proteins were incubated with 2 μg of mouse monoclonal antibody raised against SNAP-25 (Santa Cruz, sc-390814) or Syntaxin 1a (MilliporeSigma, SAB4502894) overnight, followed by incubation with 40 μl of 50% protein AG Magnetic agarose beads (Thermo Fisher., Cat. No. 78610) slurry for 1 h. The beads were washed with the binding/wash buffer (PBS with 150 mM NaCl) for 5 min at 4° C. with gentle agitation twice, then washed with deionized water once. The proteins were eluted with 100 μL of 0.1 M glycine, pH 2.2 and neutralized with 25 μL of 1M phosphate buffer, pH 7.5. Porosome reconstitution into Neuro2a APPwt and Neuro 2a APPswe cells in culture was achieved by exposing 0.125 μg/mL porosomes isolated from Neuro 2a wt cells.

### Western Blot Analysis on Cell Homogenates and Isolated Porosomes

Total cell homogenates (TH) and porosomes immuno-isolated (IP) from expanded WT (Control) mouse neuroblastoma cell culture and experimental Alzheimer's WT-APP and Sew-APP cultures were subjected to SDS-PAGE and Western Blot analysis. 20 ug of proteins (TH) and 20 μl of isolated neuronal porosomes in Laemmli buffer were resolved in a 12.5% SDS-PAGE, followed by electrotransfer to 0.2-mm nitrocellulose (NC) membrane. The NC membrane was incubated for 1 hour at RT in blocking buffer (5% nonfat milk in PBS [pH 7.4] containing 0.1% Triton X-100 and 0.02% NaN₃) and immunoblotted for 2 hours at RT with antibodies to SNAP-25 (1:1000, MilliporeSigma™, S-9684) and GAPDH (1:3,000, MilliporeSigma™, SAB600208). Anti-rabbit horseradish peroxidase secondary antibody conjugates were used (1:5,000, Cell Signaling Technology™ Cat. No. 7074), and then developed with SuperSignal™ West Femto Maximum Sensitivity Substrate (Thermo Fisher™, Cat. No. 34095) and exposed using by ChemiDoc XRT+™ image system (Bio-Rad™, Richmond, CA 94806, USA).

Immunostaining of Postmortem Hippocampus Sections from Control Subjects and AD Patients

Postmortem hippocampus sections from control subjects and AD patients (n=5 individuals/group) were stained with antibodies of (a) anti-SNAP25, anti-Neurofilament heavy chain (NF-H), and anti-NeuN; (b) anti-Syntaxin 1A, anti-Synaptophysin, and anti-NeuN; (c) anti-AT1A3, anti-MAP2, and anti-NeuN. The intensity of SNAP25, Syntaxin 1A, and AT1A3 in NeuN⁺ cells was quantified and shown in (d), (e) and (f), respectively. Hoechst was used to label nuclei. All data are presented as mean±SEM. Statistical significance was determined by unpaired Student's t-test.

### AI Driven Model Development and Biomarker Analysis

We developed a supervised autoencoder-based neural network that effectively captures complex gene expression patterns while reducing dimensionality. The model employs an encoder-decoder architecture, where the encoder learns a low-dimensional latent representation of gene expression profiles, preserving the most biologically relevant information. The decoder reconstructs the original input to ensure retention of key signal characteristics. The encoded representation is subsequently passed to a classification head that predicts disease status (AD or control). Training was performed using all genes in the dataset, allowing the network to learn a comprehensive feature set. Following initial model training, we employed SHapley Additive explanations (SHAP) (Lundberg, S. M., & Lee, S. I. (2017). A unified approach to interpreting model predictions. Advances in Neural Information Processing Systems, 30) to quantify the contribution of individual genes to classification performance. SHAP assigns an importance score to each feature, helping to identify the most influential biomarkers in distinguishing AD from control samples. The color gradients in the SHAP summary plots provide an additional layer of interpretability, illustrating how high and low expression levels of specific genes correlate with disease prediction. To evaluate the predictive value of the targeted biomarkers, we conducted a validation classification experiment where a separate model was trained exclusively on the selected genes. This model was benchmarked against 100 independent runs using randomly selected gene subsets of the same size. The goal was to determine whether the chosen biomarkers provided superior classification performance compared to arbitrary selections, thus validating their biological and diagnostic relevance.

### Dataset-Specific Results

The first dataset, GSE48350, is enriched for AD-related neuropathology and consists of 173 control and 80 disease samples. When trained on all available genes, the autoencoder model achieved 90.2% accuracy, confirming that gene expression patterns in this dataset provide strong discriminatory power. After applying SHAP to identify the influence of the targeted biomarkers (ATP1A3, ATAD3A, SNAP25, STX1A), a separate model was trained exclusively on the selected biomarkers. This model achieved an AUC of 0.71, outperforming randomly selected gene subsets, supporting the robustness of these features in disease classification. The second dataset, GSE5281, provides a broader sample distribution with 74 control and 87 disease samples. The autoencoder model achieved 87.9% accuracy when trained on the full transcriptome. SHAP analysis revealed SNAP25 as a highly influential gene. A separate classifier using only the targeted biomarkers achieved an AUC of 0.82, again surpassing models trained on randomly selected gene subsets. The SHAP-based interpretation confirms the biological relevance of these selected genes, particularly SNAP25, whose expression pattern strongly correlates with AD status. Taken together, these results demonstrate that data-driven feature selection via SHAP improves the interpretability of deep learning models applied to transcriptomic data. The ability of these biomarkers to generalize across different datasets highlights their potential utility in understanding AD pathology and developing predictive models for early detection.

### Protein-Protein Interaction Approach

To investigate the impact of amyloid beta (1-42) binding on the ATP1A3-SNAP-25-Syntaxin-1A interactions within the neuronal porosome complex, a multi-step computational workflow combining structure prediction, molecular docking, and structural analysis was employed.

### Protein Structure Prediction and Preparation

The structural models of ATP1A3, SNAP-25, and Syntaxin-1A were obtained using AlphaFold-Multimer (v2.3.1), a state-of-the-art deep learning-based protein structure prediction tool that leverages multiple sequence alignments and structural templates to generate accurate multi-chain protein complexes [1]. ATP1A3 was modeled separately for its cytosolic and extracellular domains to evaluate their respective binding interactions. The predicted structures were visually inspected and processed using PyMOL (v2.5) [2], ensuring correct domain orientation and structural integrity. Minor inconsistencies in loop positioning and atomic clashes were assessed visually, and the structures were prepared for downstream docking.

### Molecular Docking Simulations.

Protein-protein interactions were evaluated through rigid-body docking simulations using the ClusPro docking server, which applies a Fast Fourier Transform (FFT)-based sampling algorithm to predict energetically favorable binding conformations [3]. ATP1A3 was treated as the receptor in all docking experiments, while SNAP-25, Syntaxin-1A, and amyloid beta (1-42) peptide were treated as ligands in independent docking runs. The docking was conducted in two scenarios: (1) ATP1A3 cytosolic domain docking with SNAP-25 and SNAP-25-Syntaxin-1A complex, and (2) amyloid beta binding to the extracellular domain of ATP1A3, followed by intracellular docking analysis to assess structural perturbations. The docking poses were ranked based on ClusPro's default scoring function, which combines van der Waals, electrostatics, and desolvation energies.

### Binding Energy Analysis and Structural Interpretation.

Docking scores from ClusPro™ were analyzed by comparing the weighted docking scores and lowest energy conformations across different interaction scenarios. The structural alignment and visualization of docking poses were performed using PyMOL™ (v2.5) to assess conformational changes and binding interface stability. Quantitative comparisons of docking scores were interpreted to infer the effect of amyloid beta on ATP1A3-SNAP-25 interactions.

### Mouse HT-22 and Neuro2a Wt, Neuro2a APPwt and Neuro2a APPswe Cell Cultures

Mouse hippocampal HT-22 cells (MilliporeSigma™, SCC129) and mouse neuroblastoma cell line Neuro2a cells (ATCC, CCL-131) were cultured in DMEM supplemented with 10% (v/v) heat-inactivated FBS and 1% (v/v) antibiotics (100 unit/mL penicillin, 100 μg/mL streptomycin). Neuro2a cells stably overexpressing human APP wildtype (APPwt) or Swedish mutant (APPswe, K670N and M671L APP, clone Swe.10) obtained from Dr. Gopal Thinakaran (University of Chicago), were cultured as described above. Cells were plated on poly-D-lysine/laminin (P6407, Sigma-Aldrich™)-coated culture plates with or without coverslips at an appropriate cell density. All cells were maintained at 37° C. and 5% CO₂.

### Control TAT and DA1 Peptides

Control peptide TAT and DA1 peptide (Product number P103882, Lot #0P082714SF-01) were synthesized at Ontores™ (Hangzhou, China). Their purities were assessed as >90% by mass spectrometry. Lyophilized peptides were dissolved in sterile water and stored at −80° C. until use. DA1 is the first and only inhibitor that selectively binds to ATAD3A, reducing oligomerization, neuropathology, and behavioral deficits in AD mice without affecting wild-type counterparts. The peptide is particularly effective under stress or disease conditions, where oligomers accumulate and recruit Drp1. Thus, DA1-like agents offer a promising new therapeutic approach.

### Cyclic DA1 Peptide Analog

In addition to the DA1 linear peptide, we have developed cyclic DA1 analogs with enhanced metabolic stability and improved on-target efficacy for future clinical trials. Following peptide optimization and biochemical screening in AD cell cultures, a cyclic DA1 analog, DW45 has been identified. DW45 display notable stability with a half-life of >289 minutes in a mouse plasma stability assay and >216 minutes in a hepatocyte stability assay, respectively. Moreover, the analogs demonstrate stronger efficacy and on-target engagement in AD neuronal cultures, and was used in the current study.

### MTT Assay

MTT (3-(4,5-dimethylthiazol-2-yl)-2,5-diphenyltetrazolium bromide) assay is a colorimetric method that measures cell viability, proliferation, and cytotoxicity. It is based on the reduction of a yellow tetrazolium dye, MTT, into purple formazan crystals by metabolically active cells. Abeta: Amyloid beta; TAT: Transactivator of transcription; DA1 (linear peptide); WX45 (DA1 cyclic peptide)

### Immunofluorescence Cytochemistry

Cells were grown on coverslips, fixed with 4% paraformaldehyde for 20 min at room temperature, permeabilized with 0.1% Triton X-100 in PBS, and blocked with 2% normal goat serum. The cells were then incubated with the indicated primary antibodies overnight at 4° C. After washing with PBS, the cells were incubated with Alexa Fluor 488/568 or 405/568 secondary antibody (1:500; Thermo Fisher Scientific) for 2 h at room temperature. The nuclei were counterstained with DAPI (1:10,000; Sigma-Aldrich). Images of the staining were acquired using a Fluoview FV 1000 confocal microscope (Olympus).

### Reagents

Apigenin (S2262) was purchased from Selleck Chemicals LLC (Houston, TX, USA).

Preparation of oligomeric Aβ₁₋₄₂

The Aβ₁₋₄₂ (GenicBio Limited) peptides were dissolved in 1,1,1,3,3,3-hexafluoro-2-propanol (HFIP; 105228, Sigma-Aldrich™) to a final concentration of 5 mM and placed in a chemical hood overnight. The next day, HFIP was further evaporated using a SpeedVac™ concentrator for 1 h. Monomer Aβ (5 mM) was prepared by dissolving Aβ peptide in anhydrous dimethyl sulfoxide (Sigma-Aldrich). The oligomeric Aβ peptides were prepared by diluting the monomer Aβ solution in Dulbecco's Modified Eagle Medium (DMEM)/F12 and then incubating at 4° C. for 24 h.

### Human Postmortem Brain Samples

All postmortem brain samples were collected by the National Institutes of Health (NIH) NeuroBioBank (NBB; https://neuro bioba nk. nih. gov/) under the approval of the Institutional Review Boards (IRB) and the institution's Research Ethics Board. All brains were donated to the NBB by informed consent through the Brain and Tissue Repositories sites. All brain specimens donated to the NIH NBB were assessed and reviewed by board-certified neuropathologists. A standard assessment was performed to document possible neuropathologies and establish a disease condition diagnosis. In addition, postmortem blood was sampled and submitted for serology and toxicology testing. The human postmortem brain samples used in the experiments were obtained from the NBB under a material transfer agreement (MTA) between the NIH and Case Western Reserve University and listed in Table 2.

TABLE 2
Human postmortem brain samples.

| | | | | Clinical | | NFT | | | Cerebro- |
|---|---|---|---|---|---|---|---|---|---|
| | | | Brain | Final | | Braak | AD | CERAD | vascular |
| ID | Age | Gender | region | diagnosis | Dementia | Stage | pathology | score | pathology |
| HCT15H | 65 | M | Hippocampus | Unaffected | No | 0 | No | Negative | No |
| AU-19-01 | | | Cortex | control | | | indication | | |
| HCT15H | 54 | M | Hippocampus | Unaffected | No | 0 | No | Negative | No |
| AM-19-01 | | | Cortex | control | | | indication | | |
| HCT15H | 53 | M | Hippocampus | Unaffected | No | 0 | No | Negative | No |
| BY-19-01 | | | Cortex | control | | | indication | | |
| HCT16H | 56 | M | Hippocampus | Unaffected | No | 0 | No | Negative | No |
| CA-19-01 | | | Cortex | control | | | indication | | |
| HCT17H | 53 | M | Hippocampus | Unaffected | No | 0 | No | Negative | Not |
| ER-19-01 | | | Cortex | control | | | indication | | specified |
| HBGT- | 68 | F | Hippocampus | AD | Yes | IV-V | High AD | Frequent | Not |
| 19-01 | | | and Cortex | | | | neuropa- | | specified |
| | | | | | | | thological | | |
| | | | | | | | changes | | |
| | | | | | | | (A3, B3, C3) | | |
| HBBD- | 70 | M | Hippocampus | AD | Yes | V-VI | NF tangles | Frequent | Arteriolo- |
| 19-01 | | | and Cortex | | | | and neuropil | | sclerosis, |
| | | | | | | | threads, | | diffuse, mild |
| | | | | | | | amyloid | | |
| | | | | | | | accumulation, | | |
| | | | | | | | cortical | | |
| | | | | | | | atrophy, | | |
| | | | | | | | amyloid | | |
| | | | | | | | angiopathy, | | |
| HBGS- | 78 | F | Hippocampus | AD | Yes | V-VI | High AD | Frequent | Not |
| 19-01 | | | and Cortex | | | | neuropa- | | specified |
| | | | | | | | thological | | |
| | | | | | | | changes | | |
| | | | | | | | (A3, B3, C3) | | |
| HBAX- | 86 | F | Hippocampus | AD | Yes | VI | High AD | Frequent | Arteriolo- |
| 19-01 | | | and Cortex | | | | neuropa- | | sclerosis, |
| | | | | | | | thological | | diffuse, mild |
| | | | | | | | change | | to focally |
| | | | | | | | (B3, C3), | | moderate |
| | | | | | | | plaque | | |
| | | | | | | | stage, | | |
| | | | | | | | frequent | | |
| HBGE- | 88 | M | Hippocampus | AD | Yes | V-VI | High AD | Frequent | Moderate |
| 19-01 | | | and Cortex | | | | neuropa- | | small vessel |
| | | | | | | | thological | | ischemic |
| | | | | | | | changes | | disease, |
| | | | | | | | (A3, B3, C3) | | remote |
| | | | | | | | | | lacunar |
| | | | | | | | | | infarcts in |
| | | | | | | | | | parietal |
| | | | | | | | | | cortex and |
| | | | | | | | | | thalamus |

### Antibodies

Listed in Table 3 are antibodies as used herein.

TABLE 3
Antibodies

| Antibody | Manufacture | Catalog # | Dilution |
|---|---|---|---|
| Synaptophysin | Abcam ™ (Cambridge, UK) | ab32127 | 1:2000 |
| Neurofilament | Sigma-Aldrich ™ | N4142 | 1:1000 |
| (NF-H) | (St. Louis, MO, USA) | | |
| β-actin | Sigma-Aldrich ™ | A1978 | 1:10000 |
| | (St. Louis, MO, USA) | | |
| MAP2 | Cell Signaling ™ | 4542 | 1:500 |
| | (Danvers, MA, USA) | | |
| NeuN | MilliporeSigma ™ | ABN90 | 1:500 |
| | (Burlington, MA, USA) | | |
| SNAP25 | BioLegend ™ | 805002 | 1:200 |
| | (San Diego, CA, USA) | | |
| Syntaxin 1A | BioLegend ™ | 827002 | 1:500 |
| | (San Diego, CA, USA) | | |
| AT1A3 | Novus Biologicals ™ | NB300-540 | 1:200 |
| | (Centennial, CO, USA) | | |

One or more of peptides of the therapeutic peptides described herein can also be modified by natural processes, such as posttranslational processing, and/or by chemical modification techniques, which are known in the art. Modifications may occur in the peptide including the peptide backbone, the amino acid side-chains and the amino or carboxy termini. It will be appreciated that the same type of modification may be present in the same or varying degrees at several sites in a given peptide. Modifications comprise for example, without limitation, acetylation, acylation, addition of acetomidomethyl (Acm) group, ADP-ribosylation, amidation, covalent attachment to fiavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular properties, 2nd Ed., T. E. Creighton, W. H. Freeman and Company, New-York, 1993).

Peptides and/or proteins described herein may also include, for example, biologically active mutants, variants, fragments, chimeras, and analogues; fragments encompass amino acid sequences having truncations of one or more amino acids, wherein the truncation may originate from the amino terminus (N-terminus), carboxy terminus (C-terminus), or from the interior of the protein. Analogues of the invention involve an insertion or a substitution of one or more amino acids. Variants, mutants, fragments, chimeras, and analogues may function as inhibitors of the interaction of Drp1 and ATAD3A (without being restricted to the present examples).

The therapeutic peptides described herein may be prepared by methods known to those skilled in the art. The peptides and/or proteins may be prepared using recombinant DNA. For example, one preparation can include cultivating a host cell (bacterial or eukaryotic) under conditions, which provide for the expression of peptides and/or proteins within the cell.

The purification of the polypeptides may be done by affinity methods, ion exchange chromatography, size exclusion chromatography, hydrophobicity or other purification technique typically used for protein purification. The purification step can be performed under non-denaturating conditions. On the other hand, if a denaturating step is required, the protein may be renatured using techniques known in the art.

In some embodiments, the therapeutic peptides described herein can include additional residues that may be added at either terminus of a polypeptide for the purpose of providing a “linker” by which the polypeptides can be conveniently linked and/or affixed to other polypeptides, proteins, detectable moieties, labels, solid matrices, or carriers.

Amino acid residue linkers are usually at least one residue and can be 40 or more residues, more often 1 to 10 residues. Typical amino acid residues used for linking are glycine, tyrosine, cysteine, lysine, glutamic and aspartic acid, or the like. In addition, a subject polypeptide can differ by the sequence being modified by terminal-NH2 acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half-life of the polypeptides in solutions, particularly biological fluids where proteases may be present.

In some embodiments, the linker can be a flexible peptide linker that links the therapeutic peptide to other polypeptides, proteins, and/or molecules, such as detectable moieties, labels, solid matrices, or carriers. A flexible peptide linker can be about 20 or fewer amino acids in length. For example, a peptide linker can contain about 12 or fewer amino acid residues, e.g., 3, 4, 5, 6, 7, 8, 9, 10, 11, and 12. In some cases, a peptide linker comprises two or more of the following amino acids: glycine, serine, alanine, and threonine.

The therapeutic agents described herein may be modified (e.g., chemically modified). Such modification may be designed to facilitate manipulation or purification of the molecule, to increase solubility of the molecule, to facilitate administration, targeting to the desired location, to increase or decrease half-life. A number of such modifications are known in the art and can be applied by the skilled practitioner.

In the methods of treatment disclosed herein, a therapeutically effective amount of the therapeutic agent is administered to the subject to treat a disorder or mitochondrial disorder, such as a neurodegenerative disease. In one embodiment, a formulation including the therapeutic agent can be administered to the subject systemically in the period from the time of, for example, up to hours, days, and/or weeks after the disease or disorder is diagnosed.

The therapeutic agents can be delivered to a subject by any suitable route, including, for example, local and/or systemic administration. Systemic administration can include, for example, parenteral administration, such as intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally. In some embodiments, the therapeutic agent can be administered to the subject via intravenous administration using an infusion pump to deliver daily, weekly, or doses of the therapeutic agent.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein without any additional undue experimentation. It being understood that various changes may be made in the function and arrangement of elements described in an exemplary embodiment without departing from the scope of the various embodiments as set forth in the appended claims.

Since certain changes may be made in the above-described disclosure, without departing from the spirit and scope of the disclosure herein involved, it is intended that all of the subject matter of the above description shown in the accompanying drawings shall be interpreted merely as examples illustrating the inventive concept herein and shall not be construed as limiting the disclosure.

Finally, the written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A pharmaceutical composition for reducing tau protein levels in neural tissue, comprising:
a porosome complex;
a flavonoid;
and a pharmaceutically acceptable carrier,
wherein the composition is effective to reduce tau protein release in neurons.

2. The composition of claim 1 further comprising:
a synthetic blood-brain barrier traversing peptide;

3. The composition of claim 2 wherein the blood-brain barrier traversing peptide is configured as a mimic of a domain of ATP1A3 and/or Tubulin.

4. The composition of claim 1 wherein the flavonoid is apigenin.

5. The composition of claim 1 wherein the porosome protein is a neuronal porosome.

6. The composition of claim 2 wherein the synthetic blood-brain barrier traversing peptide is linear or macrocyclic DA1.

7. The composition of claim 1 wherein the composition is configured as a nanoemulsion.

8. The composition of claim 2 wherein the blood-brain barrier traversing peptide is linked to the porosome or a porosome protein.

9. The composition of claim 1 wherein the flavonoid is linked to the porosome or porosome protein.

10. The composition of claim 1 further comprising one or more porosome associated proteins.

11. A method, comprising: administering the composition of claim 1 to a mammal.


SEQUENCE LISTING (1 sequence)

SEQ ID NO: 1 - protein, 6 aa, Synthetic construct
  source 1..6: note = DA1 based linear peptide
EDKRKT